SEQUENCE LISTING (11 sequences)

SEQ ID NO: 1 - protein, 96 aa, Homo sapiens
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 2 - DNA, 20 bp, Artificial Sequence
  PCR primer
ccctggacttcgagcaagag

SEQ ID NO: 3 - DNA, 20 bp, Artificial Sequence
  PCR primer
tctccttctgcatcctgtcg

SEQ ID NO: 4 - DNA, 20 bp, Artificial Sequence
  PCR primer
atgtgctgtaccaagagttt

SEQ ID NO: 5 - DNA, 20 bp, Artificial Sequence
  PCR primer
caagtctgttttggatttgc

SEQ ID NO: 6 - DNA, 20 bp, Artificial Sequence
  PCR primer
ccattctgggcagtgagtca

SEQ ID NO: 7 - DNA, 19 bp, Artificial Sequence
  PCR primer
agcagcatcccgcagttaa

SEQ ID NO: 8 - DNA, 27 bp, Artificial Sequence
  PCR primer
agctgttggctgaaaaggtggtctatg

SEQ ID NO: 9 - DNA, 26 bp, Artificial Sequence
  PCR primer
gcgcttctggtggcccttggagtgtg

SEQ ID NO: 10 - DNA, 22 bp, Artificial Sequence
  PCR primer
atgaacgccaaggtcgtggtcg

SEQ ID NO: 11 - DNA, 19 bp, Artificial Sequence
  PCR primer
tgttgttgttcttcagccg

(19) United States
(12) Patent Application Publication
(10) Pub. No.: US 2011/0123542 A1
(43) Pub. Date: May 26, 2011

(54) CCL20-SPECIFIC ANTIBODIES FOR CANCER THERAPY

(75) Inventors: Amnon Peled, Tel Aviv (IL); Katia Beider, Jerusalem (IL)

(73) Assignee: Hadasit Medical Research Services and Development Ltd.

(21) Appl. No.: 13/000,991

(22) PCT Filed: Jun. 24, 2009
(86) PCT No.: PCT/IL09/00631

§ 371 Date: Dec. 22, 2010

Related U.S. Application Data
(60) Provisional application No. 61/075,026, filed on Jun. 24, 2008.

(51) Int. Cl. A61K 39/395; A61P 35/00; A61P 35/02

(52) U.S. Cl. 424/145.1; 424/158.1

(57) ABSTRACT

The invention is directed to the field of cancer therapy, specifically to the use of anti-CCL20 antibodies for the treatment of neoplastic disorders. The invention provides compositions and methods useful for the treatment of CCR6 and CX-CR4 expressing tumors.

### FIELD OF THE INVENTION

The invention is directed to the field of cancer therapy, specifically to antibody-based therapy for CCR6 and CXCR4 dependent tumors.

### BACKGROUND OF THE INVENTION

Chemokines, a family of small (5-20 kDa) pro-inflammatory cytokines, and their receptors, regulate a variety of immune responses to infection, inflammation and tissue repair. Primarily, chemokines are responsible for the directional migration, or chemotaxis, of lymphocytes to specific lymphoid tissues, and the recruitment of leukocytes to the sites of infection or tissue damage. In addition to their chemotactic function, chemokines are implicated in other biological events including angiogenesis, angiostasis, embryogenesis, hematopoiesis, lymphopoiesis, and HIV pathogenesis. More recently, it has been established that cancer cells exploit signaling through chemokine receptors for several key steps involved in initiation and progression of primary and metastatic cancer.

Different types of cancers express different chemokine receptors, however, only the chemokine receptor CXCR4 appears to be expressed by the majority of cancer types. Tumor cells from at least 23 different types of cancers of epithelial, mesenchymal and haematopoietic origin express CXCR4. Moreover, CXCR4 expression was found to be increased in several malignancies including gliomas, breast tumors, certain leukemia cell lines, uterine cancer, Burkitt's lymphoma, neuroblastomas, and pancreatic cancer. CXCR4 was also found to play a critical role in the progression and development of various tumors including breast, prostate and clear cell renal carcinoma (Muller et al., 2001). The importance of the CXCR4/CXCL12 pathway in tumor development was further demonstrated by neutralizing the interaction between CXCL12 and CXCR4. Using anti-CXCR4 antibodies, small molecules such as AMD3100, or by silencing the expression of CXCR4 using RNA interference technology, metastasis and progression of breast and prostate cancer in vivo in mice models was significantly impaired (Muller et al., 2001).

Recent studies have revealed the critical multifunctional role of CXCR4-CXCL12 in cancer progression. It is evident that the expression and function of the CXCR4-CXCL12 axis are tightly regulated by various biological mechanisms, most of them unknown.

CCL20, also known as macrophage inflammatory protein-3α (MIP3α), liver and activation-regulated chemokine (LARC) or exodus-1, is a 9 kDa CC-type chemokine, which is expressed constitutively at low levels by keratinocytes in epidermal layers of skin (Charbonnier et al., 1999) intestinal mucosa (Tanaka et al., 1999), liver (Hieshima et al., 1997), epithelial crypts of tonsils (Dieu et al., 1998), as well as in the epithelium of Peyer's patches in the intestine (Iwasaki, et al., 2000). While there is redundancy in human chemokine network, CCL20 is the unique chemokine ligand of its receptor CCR6. CCL20 expression has been described in a variety of human neoplasms, including colorectal, lung, pancreatic and breast human adenocarcinomas, malignant glioma, leukemia, lymphoma and melanoma.

The in vivo role of CCL20 in cancer development is controversial. Since CCL20 is a potent chemoattractant for immature dendritic cells (DCs), the most powerful antigen-presenting cells, it may serve to attract immature DCs (iDCs) to the tumor site to induce antitumor immune responses. Using this approach, Fushimi et al. demonstrated in a mouse model that intratumor injection of an adenovirus vector for gene transfer of CCL20 could suppress tumor growth (Fushimi et al., 2000). On the other hand, some evidence supports the hypothesis that CCL20 production by cancer cells promotes tumor growth and invasiveness. Contrary to the results of Fushimi et al., other groups of investigators showed that transfection of a rodent tumor cell line with CCL20 enhances tumor growth and decreases immunogenicity, despite the attraction of iDCs to the tumors (Bonnotte et al., 2004). In their model, as in human breast carcinomas which secrete high levels of CCL20 (Bell et al., 1999), DCs attracted to the tumor site did not mature.

Kleeff et al. demonstrated by immunostaining that CCL20 is overexpressed in human pancreatic carcinoma cells and in infiltrating macrophages adjacent to tumors, and suggested that CCL20 stimulates the growth and invasion of the neoplastic cells (Kleeff et al., 1999). In addition, upregulation of CCL20 was shown in human hepatocellular carcinoma tissues, and CCL20 expression level was found to correlate with tumor grade (Rubie et al., 2006).

U.S. Pat. App. Pub. No. 20050085433 relates to a composition for vaccination against tumors containing at least one tumor cell, which expresses at least one cytokine, chemokine and/or a co-stimulating molecule and an effective quantity of at least one adjuvant. The '433 application discloses that the cytokine may be inter alia MIP3α (CCL20).

WO 2008/075371, to some of the inventors of the present invention, is directed to compositions comprising T-140 peptide analogs having CXCR4 super-agonist activity and to therapeutic uses thereof for immunotherapy and vaccination. WO '371 discloses that the claimed compositions induce, in an agonist manner, secretion of MIP3α.

Although the involvement of CCL20 overexpression in either promoting or inhibiting various aspects of tumorogenicity has been investigated in different experimental models, the cumulative data are inconclusive and contradictory. To date, the in vivo role of the chemokine, in particular in the context of cancer and cancer therapy, has not been established.

Nowhere in the prior art is it demonstrated that disruption of CCL20/CCR6 interactions is effective in vivo and may be useful for treating cancer in a subject in need thereof. Indeed, antibody-based therapies directed to CCL20, involving administering CCL20-specific neutralizing antibodies, or other medicaments that specifically antagonize or neutralize CCL20, have not been reported to date. There remains an unmet medical need for providing effective therapeutic modalities for treating neoplastic disorders including CCL20 dependent tumors.

### SUMMARY OF THE INVENTION

The present invention provides composition useful for treating certain types of cancer, specifically to the use of CCL20 neutralizing agents for the treatment of CXCR4 and CCL20 dependent malignancies.

The invention is based, in part, on the surprising discovery, that neutralizing antibodies to CCL20 inhibit the in vivo growth of tumors that overexpress either CXCR4 or CCL20. In addition, it was found that CCL20 stimulated the proliferation and adhesion to collagen of various tumor cells, and that overexpression of CCL20 in tumor cells promoted growth and adhesion in vitro and increased tumor growth, spreading, invasiveness and vascularization in vivo. The present invention discloses for the first time that anti-CCL20 antibodies are effective anti cancer agents, using clinically-relevant in vivo models of colon cancer and prostate cancer.

Thus, according to a first aspect, the invention discloses for the first time the use of CCL20 neutralizing antibodies for treatment of malignancies. The present invention provides a pharmaceutical composition comprising an antibody (Ab) that specifically binds and neutralizes CCL20 for treating cancer. In various embodiments, the antibody may be an intact antibody (e.g. a polyclonal antibody or a monoclonal antibody), an antigen-binding fragment of an antibody, a recombinant antibody such as scFv, a humanized antibody etc. In one embodiment, the antibody is the known monoclonal antibody (mAb) designated MAB360 (R&D Systems, Minneapolis, Minn.), or an antibody having substantially the same specificity (a cross-reactive Ab, or an antibody comprising at least an antigen-binding fragment of MAB360).

In some embodiments, the cancer is a CCL20 dependent cancer. In another embodiment, the cancer is a CCR6 expressing cancer (i.e. expresses CCR6 on the surface of at least a portion of the cancer cells). In another embodiment, the cancer is a CXCR4 expressing cancer (i.e. expresses CXCR4 on the surface of at least a portion of the cancer cells). In another embodiment, the cancer expresses both CCR6 and CXCR4. For example, the cancer may be selected from glioma, leukemia, uterine cancer, lymphoma (e.g. Burkitt's lymphoma), neuroblastomas, pancreatic cancer (e.g. pancreatic adenocarcinomas), prostate cancer (e.g. carcinomas), clear cell renal carcinoma, colorectal, lung, and breast tumors (e.g. adenocarcinomas) and melanoma. In a particular embodiment, the cancer is prostate cancer. In another particular embodiment, the cancer is colon cancer.

In various embodiments, the composition or medicament is useful for inhibiting or reducing tumor progression, growth or vascularization, for reducing the size of an existing tumor (inducing or enhancing tumor regression) and/or for inhibiting or preventing tumor invasiveness or metastasis.

Other embodiments of the present invention are directed to therapeutic methods comprising administering to a subject in need thereof a therapeutically effective amount of an agent that specifically inhibits, antagonizes or neutralizes CCL20.

In one embodiment, there is provided a method of treating cancer, comprising administering to a subject in need thereof a therapeutically effective amount of a CCL20-neutralizing antibody.

In another embodiment, there is provided a method of inhibiting tumor growth and/or progression in a subject in need thereof, comprising administering to a subject in need thereof a therapeutically effective amount of a pharmaceutical composition comprising a CCL20-neutralizing antibody.

In another embodiment, there is provided a method of inhibiting tumor vascularization in a subject in need thereof, comprising administering to a subject in need thereof a therapeutically effective amount of a pharmaceutical composition comprising a CCL20-neutralizing antibody.

In another embodiment, there is provided a method of inducing or enhancing tumor regression (e.g. reducing tumor size or volume) in a subject in need thereof, comprising administering to a subject in need thereof a therapeutically effective amount of a pharmaceutical composition comprising a CCL20-neutralizing antibody.

In some embodiments, in the methods of the invention, the cancer or tumor is CCL20 dependent. In another embodiment, at least a portion of the cells of the cancer or tumor express CCR6. In another embodiment, at least a portion of the cells of the cancer or tumor express CXCR4. In another embodiment, at least a portion of the cells of the cancer or tumor express both CCR6 and CXCR4. For example, the cancer (or tumor) may be selected from glioma, leukemia, uterine cancer, lymphoma (e.g. Burkitt's lymphoma), neuroblastoma, pancreatic cancer (e.g. pancreatic adenocarcinoma), prostate cancer (e.g. prostate carcinoma), clear cell renal carcinoma, colon cancer, colorectal cancer, lung cancer, and breast cancer (e.g. colorectal, lung and breast adenocarcinomas) and melanoma. In a particular embodiment, the cancer is prostate cancer. In another particular embodiment, the cancer is colon cancer.

In other embodiments, the compositions and methods of the invention are useful for inhibiting or reducing CCL20-dependent tumor cell adhesion or invasiveness. Thus, in another embodiment, there is provided a method of inhibiting, preventing or reducing CCL20 dependent metastasis in a subject in need thereof, comprising administering to a subject in need thereof a therapeutically effective amount of a pharmaceutical composition comprising a CCL20-neutralizing antibody. In one embodiment, the subject has a tumor expressing CCR6 and/or CXCR4 on at least a portion of the cells of the tumor.

In another embodiment, the antibody is MAB360. In another embodiment, the antibody has the same specificity as MAB360. In another embodiment, the method comprises administering to said subject a CCL20-neutralizing agent comprising at least an antigen-binding fragment of MAB360.

Typically, the antibody is administered to the subject in the form of a pharmaceutical composition further comprising at least one pharmaceutically acceptable excipient. In some embodiments, the composition is a liquid formulation, e.g. an injectable formulation, or a formulation suitable for administration by infusion.

In another embodiment, the invention provides a pharmaceutical pack or kit containing a CCL20 neutralizing antibody of the invention, optionally formulated with at least one pharmaceutically acceptable excipient, and instructions for administering the antibody to a subject in need thereof, e.g. to a subject afflicted with cancer, as detailed herein.

Other objects, features and advantages of the present invention will become clear from the following description and drawings.

### BRIEF DESCRIPTION OF THE DRAWINGS

FIG. 1 demonstrates regulation of CCL20 expression and function. FIG. 1A Surface CCR6 expression in prostate cancer cell lines PC3, LaNCAP, 22Rv1 and DU145 evaluated by FACS. Full histograms represent mouse IgG control antibody, empty histograms represent staining with CCR6 monoclonal antibody. FIG. 1B—viability and proliferation. PC3 and PC3-CXCR4 cells were incubated with various concentration of CCL20 for 6 days. Following 3 days of incubation the medium with or without CCL20 was renewed. On day 6 the cells were harvested and viable cells were counted using PI staining and FACS analysis. In addition, in order to determine proliferation of PC3 cells, the cells were labeled with BrdU (10 μM) during the last 16 hours of incubation and processed for BrdU detection using specific anti-BrdU FITC-conjugated antibody and FACS analysis. Data is presented as mean±SD from triplicates (** P<0.05). Data is representative of two separate experiments. FIG. 1C—adhesion assay. PC3 cells either untreated or treated with various concentrations of CCL20 were placed on collagen I- or fibronectin-coated plates (10 μg/ml) for 30 minutes. Non-adherent cells were washed twice with cold PBS. Data is presented as mean±SD from triplicates (** P<0.05). In addition, PC3 cells that demonstrated increased adhesion to collagen I and fibronectin in response to stimulation with CCL20, were co-incubated with CCL20 and PTX (100 ng/ml) and were allowed to adhere to collagen I- and fibronectin-coated plates. FIG. 1D—adhesion assay. PC3-CXCR4.5 were treated and examined as described in FIG. 1C.

FIG. 2 demonstrates regulation of CCL20 expression and function in various tumor cells. FIG. 2A—CCL20 mRNA and protein expression in CCL20-transfected PC3 single-cell clones tested by semi-quantitative RT-PCR and ELISA. FIG. 2B—cell viability. PC3-CCL20 clones were seeded at 2×10⁴ cells/1 ml per well into a 24-well plate and incubated for 6 days. On day 6 the cells were harvested and viable cells were counted using PI staining and FACS analysis. Data is presented as mean±SD from triplicates (** P<0.05). FIG. 2C—adhesion assay. PC3-CCL20 single-cell clones were allowed to adhere to collagen I- and fibronectin-coated plates for 30 minutes. Data is presented as mean±SD from triplicates (** P<0.05). FIG. 2D—CCL20 secretion. Leukemic cell lines NB4 (top left) and HL60 (top right), primary human leukemic blasts (bottom left) and HT-29 cells (bottom right) were incubated with various concentrations of CXCL12 for 48 hours. CCL20 secretion to culture medium was assessed using ELISA method. FIG. 2E—CCR6 mRNA expression in leukemic cell lines NB4 and HL60 and colon cancer HT-29 cells assessed by semi-quantitative RT-PCR. β-actin confirmed comparable loading of RT-PCR products in each lane. FIG. 2F—adhesion assay. HL60 (left) and HT-29 (right) cells either untreated or treated with various concentrations of CCL20 were placed on collagen I coated plates (10 μg/ml) for 30 minutes. Non-adherent cells were washed twice with cold PBS. Adherent cells were collected in 300 μl FACS buffer with 5 mM EDTA and counted by FACS. Data is presented as mean±SD from triplicates (** P<0.05). Data is representative of three separate experiments.

FIG. 3 shows that CCL20 regulates CXCR4 dependent and independent growth of tumor cells. FIG. 3A—Effect of CCL20 stable expression on prostate tumor growth. PC3-CCL20.30, PC3-CCL20.10 and PC3-mock transfected cells (5×10⁶/mouse) are shown. Results are representative of three independent experiments. Data is presented as mean±SE from five mice. FIG. 3B—H&E staining of paraffin-embedded tumor tissue sections derived from PC3-mock and PC3-CCL20.30 tumors on day 48. Black arrows sign non-invasive borders of PC3-mock tumor (panel a), small blood vessel in PC3-mock tumor (panel b), aberrant blood vessels in PC3-CCL20.30 tumor (panels c, d), original magnification of ×200 is shown. FIG. 3C—Vessel functionality (ΔSo₂) was measured by fMRI. Functionality of the vasculature was tested during inhalation of air-CO₂ and carbogen (95% oxygen+5% CO₂) in mice implanted with PC3-mock cells or with PC3-CCL20.30 cells. ΔSo₂ values from PC3-mock cells and PC3-CCL20.30 are shown. The mean±SD values of ΔSo₂ from 9 mice from the PC3-CCL20.30 group and 5 mice from the PC3-mock group are shown (four slices/mouse; 6C p<0.001). FIG. 3D—Adhesion of PC3-CCL20.30 cells to collagen I. PC3-CCL20.30 cells either umstimulated or stimulated with 50 ng/ml of CCL20 with or without co-incubation with neutralizing anti-CCL20 antibodies (aCCL20, 10 μg/ml) are shown. Data is presented as mean±SD from triplicates (** P<0.016). FIG. 3E—effect of neutralizing anti-CCL20 antibodies on tumor size. PC3-CCL20.30 cells (5×10⁶/mouse) were injected subcutaneously into SCID/beige mice. Mice were treated with anti-human CCL20 antibodies or isotype control antibodies (IgG-treated), 20 μg of antibody per injection, three times a week, during four weeks. Tumor size (cm²) was measured once a week using caliper. Results are representative of two independent experiments with ten mice in each group. Data is presented as mean±SE from ten mice. FIG. 3F—effect of neutralizing anti-CCL20 antibodies on tumor weight. At day 66 subcutaneous tumors were removed, measured and weighted. Data is presented as mean±SE from ten mice in each group (** P<0.0002). FIG. 3G—effect of neutralizing anti-CCL20 antibodies on tumor size and weight. PC3-CXCR4.5 cells (5×10⁶/mouse) were injected subcutaneously into SCID/beige mice. Mice were treated with anti-human CCL20 antibodies or isotype control antibodies, 20 μg of antibody per injection, three times a week, during four weeks. At day 55 animals were sacrificed and subcutaneous tumors were measured and weighted. Data is presented as mean±SE from ten mice in each group (** P<0.0027). FIG. 3H—effect of neutralizing anti-CCL20 antibodies on tumor size and weight. HT-29 cells (2×10⁶/mouse) were injected subcutaneously into nude mice. Mice were treated with anti-human CCL20 antibodies or isotype control antibodies, 20 μg of antibody per injection, five times a week, during two weeks. At day 17 mice were sacrificed and subcutaneous tumors were measured and weighted. Data is presented as mean±SE from ten mice in each group (** P<0.0002).

FIG. 4 depicts CCL20 and CCR6 expression in prostate cancer cell lines, in primary prostate tumor tissue and in normal prostate tissue. Immunohistostaining of prostate cancer and normal specimens using the polyclonal antibody for CCL20 and the monoclonal antibody 140706 for CCR6. Original magnification of ×400 is shown. CCL20 and CCR6 expression was observed in endothelial and fibromuscular cells of prostate samples (signed with black arrows).

### DETAILED DESCRIPTION OF THE INVENTION

The invention is directed to the use of anti-CCL20 antibodies for the treatment of neoplastic disorders. The invention provides compositions and methods useful for the treatment of CCR6 and CXCR4 expressing tumors.

According to a first aspect of the present invention, there is provided a method for treating a CCL20 dependent cancer in a subject in need thereof, comprising administering to the subject a therapeutically effective amount of a pharmaceutical composition comprising a CCL20-neutralizing antibody. In one embodiment, the method is useful for inhibiting tumor growth and/or progression in the subject. In another embodiment, the method is useful for inhibiting tumor vascularization in the subject. In another embodiment, the method is useful for inducing or enhancing tumor regression in the subject.

In another embodiment, at least a portion of the cells of the cancer express CCR6. In another embodiment, at least a portion of the cells of the cancer express CXCR4. In yet another embodiment, at least a portion of the cells of the cancer are characterized by surface expression of CCR6 and CXCR4.

According to another embodiment, the cancer is selected from glioma, leukemia, uterine cancer, lymphoma, neuroblastomas, pancreatic cancer, prostate cancer, clear cell renal carcinoma, colorectal, lung, and breast tumors and melanoma. In one particular embodiment, the cancer is prostate cancer. In another particular embodiment, the cancer is colon cancer.

In another embodiment, the antibody is MAB360. In various other embodiments, the antibody has the same specificity as MAB360, or wherein said antibody has at least an antigen-binding fragment of MAB360.

In other embodiments, the antibody is administered to the subject by injection or infusion.

In another aspect, the invention provides a method of inhibiting, preventing or reducing metastasis of a CCL20 dependent tumor in a subject in need thereof, comprising administering to a subject in need thereof a therapeutically effective amount of a CCL20-neutralizing antibody.

In one embodiment, the subject has a tumor expressing CCR6 and/or CXCR4 on at least a portion of the cells of the tumor.

In another embodiment, the antibody is MAB360. In other embodiments, the antibody has the same specificity as MAB360, or has at least an antigen-binding fragment of MAB360.

In another embodiment, the antibody is administered to the subject by injection or infusion.

In another aspect, there is provided a pharmaceutical composition comprising a CCL20 neutralizing antibody for the treatment of cancer.

In one embodiment, the cancer is a CCL20 dependent cancer. In other embodiments, the cancer is selected from glioma, leukemia, uterine cancer, lymphoma, neuroblastomas, pancreatic cancer, prostate cancer, clear cell renal carcinoma, colorectal, lung, and breast tumors and melanoma.

In various other embodiments, the medicament is useful for inhibiting or reducing tumor progression, growth or vascularization, for reducing the size of an existing tumor and/or for inhibiting or preventing tumor invasiveness or metastasis.

According to further embodiments, the antibody is MAB360, an antibody having the same specificity as MAB360, or an antibody having at least an antigen-binding fragment of MAB360.

According to additional embodiments, the medicament is an injectable formulation or a formulation suitable for administration via infusion.

In another aspect, the invention provides a kit comprising a CCL20 neutralizing antibody, optionally formulated with at least one pharmaceutically acceptable excipient, and instructions for administering the antibody to a subject afflicted with cancer

In one embodiment, the cancer is a CCL20 dependent cancer. In other embodiments, the cancer is selected from glioma, leukemia, uterine cancer, lymphoma, neuroblastomas, pancreatic cancer, prostate cancer, clear cell renal carcinoma, colorectal, lung, and breast tumors and melanoma.

In other embodiments, the antibody is MAB360, an antibody having the same specificity as MAB360, or an antibody having at least an antigen-binding fragment of MAB360.

Antibodies

The present invention relates to agents that specifically antagonize, neutralize or otherwise inhibit or interfere with CCL20/CCR6 interactions in CCL20-dependent cancer cells, thereby inhibiting the tumorogenicity of said cells. According to various embodiments, these agents are antibodies, particularly CCL20-specific antibodies.

The term “antibody” (or Ab) as used herein refers to an immunoglobulin or fragment thereof, and encompasses any molecule (e.g. polypeptide) comprising an antigen-binding fragment or an antigen-binding domain. The term includes but is not limited to polyclonal, monoclonal, humanized, human, single-chain, chimeric, synthetic, recombinant, hybrid, mutated, grafted, and in vitro generated antibodies. Unless preceded by the word “intact”, the term “antibody” includes antibody fragments such as Fab, Fab′ F(ab′)₂, Fv and other antibody fragments that retain antigen-binding function. Typically, such fragments would comprise an antigen-binding domain. Such molecules may be provided by any known technique, including, but not limited to, enzymatic cleavage, peptide synthesis or recombinant techniques.

Antibodies, or immunoglobulins, comprise two heavy chains linked together by disulfide bonds and two light chains, each light chain being linked to a respective heavy chain by disulfide bonds in a “Y” shaped configuration. Proteolytic digestion of an antibody yields Fv (Fragment variable) and Fc (fragment crystalline) domains. The antigen binding domains, Fab′, include regions where the polypeptide sequence varies. The term F (ab′)₂ represents two Fab′ arms linked together by disulfide bonds. The central axis of the antibody is termed the Fc fragment, and is known to mediate phagocytosis, trigger inflammation and target Ig to particular tissues; the Fc portion is also important in complement activation. Each heavy chain has at one end a variable domain (V_{H}) followed by a number of constant domains (C_{H}). Each light chain has a variable domain (V_{L}) at one end and a constant domain (CO at its other end, the light chain variable domain being aligned with the variable domain of the heavy chain and the light chain constant domain being aligned with the first constant domain of the heavy chain (C_{H1}).

The variable domains of each pair of light and heavy chains form the antigen binding site. The domains on the light and heavy chains have the same general structure and each domain comprises four framework regions, whose sequences are relatively conserved, joined by three hypervariable domains known as complementarity determining regions (CDR₁₋₃). These domains contribute to the specificity and affinity of the antigen binding site.

The terms “antigen-binding domain” and “antigen-binding fragment” refer to a part of an antibody molecule that comprises-amino acids responsible for the specific binding between antibody and antigen. The part of the antigen that is specifically recognized and bound by the antibody is referred to as the “epitope”. An antigen-binding domain may comprise an antibody light chain variable region (V_{L}) and an antibody heavy chain variable region (V_{H}); however, it does not have to comprise both. Fd fragments, for example, have two V_{H} regions and often retain some antigen-binding function of the intact antigen-binding domain.

Epitopes or antigenic determinants usually consist of chemically active surface groupings of molecules such as amino acids or sugar side chains and have specific three-dimensional structural characteristics as well as specific charge characteristics.

An “antigen” is a molecule or a portion of a molecule capable of being bound by an antibody, which is additionally capable of inducing an animal to produce antibodies capable of binding to an epitope of that antigen. An antigen may have one or more than one epitope. The specific reaction referred to herein is meant to indicate that the antigen will react, in a highly selective manner, with its corresponding antibody and not with the multitude of other antibodies which may be evoked by other antigens.

The isotype of the heavy chain (gamma, alpha, delta, epsilon or mu) determines immunoglobulin class (IgG, IgA, IgD, IgE or IgM, respectively). The light chain is either of two isotypes (kappa, or lambda) found in all antibody classes.

Single chain antibodies fall within the scope of the present invention. Single chain antibodies can be single chain composite polypeptides having antigen binding capabilities and comprising amino acid sequences homologous or analogous to the variable regions of an immunoglobulin light and heavy chain (linked V_{H}-V_{L} or single chain Fv (ScFv)). Both V_{H} and V_{L} may copy natural monoclonal antibody sequences or one or both of the chains may comprise a CDR-FR construct of the type described in U.S. Pat. No. 5,091,513, the entire contents of which are hereby incorporated herein by reference. The separate polypeptides analogous to the variable regions of the light and heavy chains are held together by a polypeptide linker. Methods of production of such single chain antibodies, particularly where the DNA encoding the polypeptide structures of the V_{H} and V_{L} chains are known, may be accomplished in accordance with the methods described, for example, in U.S. Pat. Nos. 4,946,778, 5,091,513 and 5,096,815, the entire contents of each of which are hereby incorporated herein by reference.

Fab miniantibodies (see WO 93/15210, WO 96/13583 and WO 96/37621, the entire contents of which are incorporated herein by reference) and chimeric or single-chain antibodies incorporating such reactive fraction, as well as any other type of molecule or cell in which such antibody reactive fraction has been physically inserted, such as a chimeric T-cell receptor, are also encompassed within certain embodiments of the present invention. Such molecules may be provided by any known technique, including, but not limited to, enzymatic cleavage, peptide synthesis or recombinant techniques.

Methods of generating monoclonal and polyclonal antibodies are well known in the art. Antibodies may be generated via any one of several known methods, which may employ induction of in vivo production of antibody molecules, screening of immunoglobulin libraries, or generation of monoclonal antibody molecules by continuous cell lines in culture. These include, but are not limited to, the hybridoma technique, the human B-cell hybridoma technique, and the Epstein-Barr virus (EBV)-hybridoma technique.

In cases where target antigens are too small to elicit an adequate immunogenic response when generating antibodies in vivo, such antigens (referred to as “haptens”) can be coupled to antigenically neutral carriers such as keyhole limpet hemocyanin (KLH) or serum albumin (e.g., bovine serum albumin (BSA)) carriers (see, for example, U.S. Pat. Nos. 5,189,178 and 5,239,078). Coupling a hapten to a carrier can be effected using methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill., USA. The resulting immunogenic complex can then be injected into suitable mammalian subjects such as mice, rabbits, and others. Suitable protocols involve repeated injection of the immunogen in the presence of adjuvants according to a schedule designed to boost production of antibodies in the serum. The titers of the immune serum can readily be measured using immunoassay procedures which are well known in the art.

The antisera obtained can be used directly (e.g. as diluted sera or as purified polyclonal antibodies), or monoclonal antibodies may be obtained, as described herein.

A monoclonal antibody (mAb) is a substantially homogeneous population of antibodies to a specific antigen. mAbs may be obtained by methods known to those skilled in the art. See, for example U.S. Pat. No. 4,376,110; Ausubel et al (“Current Protocols in Molecular Biology,” Volumes I-III, John Wiley & Sons, Baltimore, Md., 1994). A hybridoma producing a mAb may be cultivated in vitro or in vivo. High titers of mAbs can be obtained in in vivo production where cells from the individual hybridomas are injected intraperitoneally into pristine-primed Balb/c mice to produce ascites fluid containing high concentrations of the desired mAbs. MAbs of isotype IgM or IgG may be purified from such ascites fluids, or from culture supernatants, using column chromatography methods well known to those of skill in the art.

Antibody fragments may be obtained using methods well known in the art. (See, for example, Harlow, E. and Lane, D. (1988). Antibodies: A Laboratory Manual, Cold Spring Harbor Laboratory, New York). For example, antibody fragments according to the present invention can be prepared by proteolytic hydrolysis of the antibody or by expression in E. coli or mammalian cells (e.g., Chinese hamster ovary (CHO) cell culture or other protein expression systems) of DNA encoding the fragment.

Alternatively, antibody fragments can be obtained by pepsin or papain digestion of whole antibodies by conventional methods. As described hereinabove, (Fab′)₂ antibody fragments can be produced by enzymatic cleavage of antibodies with pepsin to provide a 5S fragment. This fragment can be further cleaved using a thiol reducing agent, and optionally a blocking group for the sulfhydryl groups resulting from cleavage of disulfide linkages, to produce 3.5S Fab′ monovalent fragments. Alternatively, enzymatic cleavage using pepsin produces two monovalent Fab′ fragments and an Fc fragment directly. Ample guidance for practicing such methods is provided in the literature of the art (for example, refer to: U.S. Pat. Nos. 4,036,945 and 4,331,647). Other methods of cleaving antibodies, such as separation of heavy chains to form monovalent light-heavy chain fragments, further cleavage of fragments, or other enzymatic, chemical, or genetic techniques may also be used, so long as the fragments retain the ability to bind to the antigen that is recognized by the intact antibody.

Besides the conventional method of raising antibodies in vivo, antibodies can be generated in vitro using phage display technology. Such a production of recombinant antibodies is much faster compared to conventional antibody production and they can be generated against an enormous number of antigens. In contrast, in the conventional method, certain antigens prove to be non-immunogenic or extremely toxic, and therefore cannot be used to generate antibodies in animals. Moreover, affinity maturation (i.e., increasing the affinity and specificity) of recombinant antibodies is very simple and relatively fast. Finally, large numbers of different antibodies against a specific antigen can be generated in one selection procedure. To generate recombinant monoclonal antibodies one can use various methods all based on phage display libraries to generate a large pool of antibodies with different antigen recognition sites. Such a library can be made in several ways: One can generate a synthetic repertoire by cloning synthetic CDR₃ regions in a pool of heavy chaingermline genes and thus generating a large antibody repertoire, from which recombinant antibody fragments with various specificities can be selected. One can use the lymphocyte pool of humans as starting material for the construction of an antibody library. It is possible to construct naive repertoires of human IgM antibodies and thus create a human library of large diversity. This method has been widely used successfully to select a large number of antibodies against different antigens. Protocols for bacteriophage library construction and selection of recombinant antibodies are provided in the well-known reference text Current Protocols in Immunology, Colligan et al (Eds.), John Wiley & Sons, Inc. (1992-2000), Chapter 17, Section 17.1.

As described hereinabove, an Fv is composed of paired heavy chain variable and light chain variable domains. This association may be noncovalent. Alternatively, as described hereinabove, the variable domains may be linked to generate a single-chain Fv by an intermolecular disulfide bond, or alternately such chains may be cross-linked by chemicals such as glutaraldehyde.

Preferably, the Fv is a single-chain Fv. Single-chain Fvs are prepared by constructing a structural gene comprising DNA sequences encoding the heavy chain variable and light chain variable domains connected by an oligonucleotide encoding a peptide linker. The structural gene is inserted into an expression vector, which is subsequently introduced into a host cell such as E. coli. The recombinant host cells synthesize a single polypeptide chain with a linker peptide bridging the two variable domains. Ample guidance for producing single-chain Fvs is provided in the literature of the art. Improved bivalent miniantibodies, with identical avidity as whole antibodies, may be produced by high cell density fermentation of Escherichia coli. (U.S. Pat. No. 4,946,778).

Isolated complementarity-determining region peptides can be obtained by constructing genes encoding the CDR of an antibody of interest. Such genes may be prepared, for example, by RT-PCR of the mRNA of an antibody-producing cell. Ample guidance for practicing such methods is provided in the literature of the art.

The term “human antibody” includes antibodies having variable and constant regions corresponding substantially to human germline immunoglobulin sequences known in the art. The human antibodies of the invention may include amino acid residues not encoded by human germline immunoglobulin sequences (e.g., mutations introduced by random or site-specific mutagenesis in vitro or by somatic mutation in vivo), for example in the CDRs, and in particular, CDR3. The human antibody can have at least one, two, three, four, five, or more positions replaced with an amino acid residue that is not encoded by the human germline immunoglobulin sequence.

Chimeric antibodies are molecules, the different portions of which are derived from different animal species, such as those having a variable region derived from a murine mAb and a human immunoglobulin constant region. Antibodies which have variable region framework residues substantially from human antibody (termed an acceptor antibody) and complementarity determining regions substantially from a mouse antibody (termed a donor antibody) are also referred to as humanized antibodies. Chimeric antibodies are primarily used to reduce immunogenicity in application and to increase yields in production, for example, where murine mAbs have higher yields from hybridomas but higher immunogenicity in humans, such that human/murine chimeric mAbs are used. Chimeric antibodies and methods for their production are known in the art (e.g. European Patent Applications 125023, 171496, 173494, 184187, 173494, PCT patent applications WO 86/01533, WO 97/02671, WO 90/07861, WO 92/22653 and U.S. Pat. Nos. 5,693,762, 5,693,761, 5,585,089, 5,530,101 and 5,225,539). Additionally, CDR grafting may be performed to alter certain properties of the antibody molecule including affinity or specificity. A non-limiting example of CDR grafting is disclosed in U.S. Pat. No. 5,225,539. Further methods for producing chimeric antibodies are described, for example, in U.S. Pat. No. 4,816,567. These references are hereby incorporated by reference.

It will be appreciated that for human therapy, humanized antibodies are preferably used. Humanized forms of non-human (e.g., murine) antibodies are genetically engineered chimeric antibodies or antibody fragments having (preferably minimal) portions derived from non-human antibodies. Humanized antibodies include antibodies in which the CDRs of a human antibody (recipient antibody) are replaced by residues from a CDR of a non-human species (donor antibody), such as mouse, rat, or rabbit, having the desired functionality. In some instances, the Fv framework residues of the human antibody are replaced by corresponding non-human residues. Humanized antibodies may also comprise residues found neither in the recipient antibody nor in the imported CDR or framework sequences. In general, the humanized antibody will comprise substantially all of at least one, and typically two, variable domains, in which all or substantially all of the CDRs correspond to those of a non-human antibody and all or substantially all of the framework regions correspond to those of a relevant human consensus sequence. Humanized antibodies optimally also include at least a portion of an antibody constant region, such as an Fc region, typically derived from a human antibody.

Methods for humanizing non-human antibodies are well known in the art. Generally, a humanized antibody has one or more amino acid residues introduced into it from a source which is non-human. These non-human amino acid residues are often referred to as imported residues, which are typically taken from an imported variable domain. Humanization can be performed as is known in the art (see, for example: U.S. Pat. No. 4,816,567), by substituting human CDRs with corresponding rodent CDRs. Accordingly, humanized antibodies are chimeric antibodies, wherein substantially less than an intact human variable domain has been substituted by the corresponding sequence from a non-human species. In practice, humanized antibodies may be typically human antibodies in which some CDR residues and possibly some framework residues are substituted by residues from analogous sites in rodent antibodies.

Human antibodies can also be produced using various additional techniques known in the art, including phage-display libraries. Humanized antibodies can also be created by introducing sequences encoding human immunoglobulin loci into transgenic animals, e.g., into mice in which the endogenous immunoglobulin genes have been partially or completely inactivated. Upon antigenic challenge, human antibody production is observed in such animals which closely resembles that seen in humans in all respects, including gene rearrangement, chain assembly, and antibody repertoire. Ample guidance for practicing such an approach is provided in the literature of the art (for example, refer to: U.S. Pat. Nos. 5,545,807; 5,545,806; 5,569,825; 5,625,126; 5,633,425; and 5,661,016).

After antibodies have been obtained, they may be tested for activity, for example via enzyme-linked immunosorbent assay (ELISA).

In various embodiments, the antibodies of the present invention are anti-CCL20 antibodies, i.e. Abs that specifically bind to CCL20. The terms “specific binding” or “specifically binds” refers to two molecules forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the association constant K_{A} is higher than 10⁶ M⁻¹. If necessary, nonspecific binding can be reduced without substantially affecting specific binding by varying the binding conditions. The appropriate binding conditions, such as concentration of antibodies, ionic strength of the solution, temperature, time allowed for binding, concentration of a blocking agent (e.g., serum albumin, milk casein), etc., may be optimized by a skilled artisan using routine techniques. The term “specifically bind” as used herein may further indicate that the binding of an antibody to an antigen is not competitively inhibited by the presence of non-related molecules. Conveniently, detection of the capacity of an antibody to specifically bind an antigen, e.g. CCL20, may be performed by quantifying specific antigen-antibody complex formation (e.g. by ELISA).

In some embodiments, the present invention is directed to CCL20-neutralizing antibodies. A “neutralizing antibody” as used herein refers to a molecule having an antigen binding site to a target molecule, e.g. a chemokine, which is capable of reducing or inhibiting (blocking) activity or signaling mediated by the chemokine and/or the respective chemokine receptor. This activity or signaling is conveniently determined by in vivo or in vitro assays, as per the specification. In various embodiments, CCL20-neutralizing antibodies of the invention are anti-CCL20 antibodies that inhibit CCL20 activity. The phrase “inhibit” or “antagonize” CCL20 activity and its cognates refers to a reduction, inhibition, or otherwise diminution of at least one activity of CCL20 due to binding an anti-CCL20 antibody, wherein the reduction is relative to the activity of CCL20 in the absence of the same antibody. The activity can be measured using any technique known in the art, including, for example, as described in the Examples. Inhibition or antagonism does not necessarily indicate a total elimination of the CCL20 polypeptide biological activity. A reduction in activity may be about 10%, 20%, 30%, 40%, 50%, 60%, 70%, 80%, 90%, or more.

The term “CCL20” refers to a cytokine (which may be mammalian) capable of binding to CCR6 receptor, and has at least one of the following features: (1) an amino acid sequence of a naturally occurring mammalian CCL20 polypeptide (full length or mature form) or a fragment thereof, e.g., an amino acid sequence shown as SEQ ID NO:1, as follows:

MCCTKSLLLA ALMSVLLLHL CGESEAASNF DCCLGYTDRI
LHPKFIVGFT RQLANEGCDI NAIIFHTKKK LSVCANPKQT
WVKYIVRLLS KKVKNM

(genebank no. P78556, human CCL20); (2) an amino acid sequence substantially identical to, e.g., at least 85%, 90%, 95%, 96%, 97%, 98%, 99% identical to, an amino acid sequence shown as SEQ ID NO: 1 or a fragment thereof; (3); an amino acid sequence encoded by a nucleotide sequence degenerate to a naturally occurring CCL20 nucleotide sequence or a fragment thereof; or (6) a nucleotide sequence that hybridizes to one of the foregoing nucleotide sequences under stringent conditions, e.g., highly stringent conditions. The CCL20 may bind to CCR6 receptor of mammalian origin, e.g., human CCR6.

In one particular embodiment, the antibody is the known mAb designated MAB360 (R&D Systems, Minneapolis, Minn.). This antibody is a mouse antibody of the IgG1 isotype generated against recombinant human CCL20 (clone 67310, cat. No. MAB360). It should be appreciated, that for human use, adequately purified antibody preparations (sufficiently sterile and free from toxic agents or other impurities) are used, as known in the art. In other specific embodiments, the antibody has substantially the same specificity as MAB360. For example, the antibody may contain an antigen-binding fragment of MAB360, or it may contain an antigen-binding fragment which is not identical to that of MAB360, but recognizes the same epitope (or a substantially overlapping epitope) in a specific manner. In one embodiment, the antibody is characterized in that it competes with MAB360 on binding to CCL20. For example, in the presence of the antibody at the same concentration, the binding of MAB360 to CCL20 is reduced by at least 50%. In various embodiments, the antibody will bind to CCL20 with equivalent, better or up to two orders of magnitude weaker affinity.

Pharmaceutical Compositions

According to another embodiment, the present invention provides a pharmaceutical composition comprising as an active ingredient a CCL20-neutralizing or antagonizing agent according to the invention, for use in therapy. Said compositions may be in any pharmaceutical form suitable for administration to a patient, including but not limited to solutions, suspensions, lyophilized powders for reconstitution with a suitable vehicle or dilution prior to usage, capsules, tablets, sustained-release formulations and the like. The compositions may comprise a therapeutically effective amount of an antibody of the present invention, preferably in purified form, and a pharmaceutical excipient. As used herein, “pharmaceutical excipient” includes solvents, dispersion media, coatings, antibacterial and antifungal agents, isotonic and absorption delaying agents etc. and combinations thereof, which are compatible with pharmaceutical administration. The compositions may also contain other active compounds providing supplemental, additional, or enhanced therapeutic functions. In another embodiment, the composition consists essentially of a CCL20-neutralizing antibody and one or more pharmaceutical excipients. In another embodiment, the composition consists of a CCL20-neutralizing antibody and one or more pharmaceutical excipients.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes. A pharmaceutical composition of the invention is formulated to be compatible with its intended route of administration. Methods to accomplish the administration are known to those of ordinary skill in the art. Examples of suitable excipients and modes for formulating the compositions are described in the latest edition of “Remington's Pharmaceutical Sciences” by E. W. Martin.

Pharmaceutical compositions according to the invention are typically liquid formulations suitable for injection or infusion. Examples of administration of a pharmaceutical composition include oral ingestion, inhalation, intravenous and continues infusion, intraperitoneal, intramuscular, intracavity, subcutaneous, cutaneous, or transdermal administration. According to certain particular embodiments, the compositions are suitable for intralesional (e.g. intratumoral) administration.

For example, saline solutions and aqueous dextrose and glycerol solutions can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Solutions or suspensions used for intravenous administration typically include a carrier such as physiological saline, bacteriostatic water, Cremophor EL™ (BΔSF, Parsippany, N.J.), ethanol, or polyol. In all cases, the composition must be sterile and fluid for easy syringability. Proper fluidity can often be obtained using lecithin or surfactants. The composition must also be stable under the conditions of manufacture and storage. Prevention of microorganisms can be achieved with antibacterial and antifungal agents, e.g., parabens, chlorobutanol, phenol, ascorbic acid, thimerosal, etc. In many cases, isotonic agents (sugar), polyalcohols (mannitol and sorbitol), or sodium chloride may be included in the composition. Prolonged absorption of the composition can be accomplished by adding an agent which delays absorption, e.g., aluminum monostearate and gelatin. Where necessary, the composition may also include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Oral compositions include an inert diluent or edible carrier. The composition can be enclosed in gelatin or compressed into tablets. For the purpose of oral administration, the antibodies can be incorporated with excipients and placed in tablets, troches, or capsules. Pharmaceutically compatible binding agents or adjuvant materials can be included in the composition. The tablets, troches, and capsules, may optionally contain a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; or a sweetening agent or a flavoring agent.

The composition may also be administered by a transmucosal or transdermal route. For example, antibodies that comprise an Fc portion may be capable of crossing mucous membranes in the intestine, mouth, or lungs (via Fc receptors). Transmucosal administration can be accomplished through the use of lozenges, nasal sprays, inhalers, or suppositories. Transdermal administration can also be accomplished through the use of a composition containing ointments, salves, gels, or creams known in the art. For transmucosal or transdermal administration, penetrants appropriate to the barrier to be permeated are used. For administration by inhalation, the antibodies are delivered in an aerosol spray from a pressured container or dispenser, which contains a propellant (e.g., liquid or gas) or a nebulizer. The composition can be formulated as a suppository, with traditional binders and carriers such as triglycerides.

Solutions or suspensions used for intradermal or subcutaneous application typically include at least one of the following components: a sterile diluent such as water, saline solution, fixed oils, polyethylene glycol, glycerine, propylene glycol, or other synthetic solvent; antibacterial agents such as benzyl alcohol or methyl parabens; antioxidants such as ascorbic acid or sodium bisulfite; chelating agents such as ethylenediaminetetraacetic acid (EDTA); buffers such as acetate, citrate, or phosphate; and tonicity agents such as sodium chloride or dextrose. The pH can be adjusted with acids or bases. Such preparations may be enclosed in ampoules, disposable syringes, or multiple dose vials.

In certain embodiments, the antibodies of this invention are prepared with carriers to protect the antibodies against rapid elimination from the body. Biodegradable polymers (e.g., ethylene vinyl acetate, polyanhydrides, polyglycolic acid, collagen, polyorthoesters, and polylactic acid) are often used. Methods for the preparation of such formulations are known by those skilled in the art. Liposomal suspensions can be used as pharmaceutically acceptable carriers too. The liposomes can be prepared according to established methods known in the art (U.S. Pat. No. 4,522,811).

In addition, the antibodies of the present invention may be administered with various effector molecules such as heterologous polypeptides, drugs, radionucleotides, or toxins.

The pharmaceutical compositions may also be included in a container, pack, or dispenser and optionally instructions for administration. For example, the kit may contain instructions for administering the composition to a subject afflicted with cancer, as detailed herein.

Pharmaceutical compositions suitable for use in context of the present invention include compositions wherein the active ingredients are contained in an amount effective to achieve the intended purpose. All formulations for administration should be in dosages suitable for the chosen route of administration. More specifically, a “therapeutically effective” dose means an amount of a compound effective to prevent, alleviate or ameliorate symptoms of a disease of the subject being treated. Determination of a therapeutically effective amount is well within the capability of those skilled in the art, especially in light of the detailed disclosure and Examples provided herein.

In certain circumstances, it may be advantageous to formulate compositions in unit dosage form for ease of administration and uniformity of dosage. Unit dosage form as used herein refers to physically discrete units suited for the patient. Each unit dosage contains a predetermined quantity of e.g. an antibody calculated to produce a therapeutic effect in association with the carrier. The unit dosage depends on the characteristics of the agent (e.g. antibodies) and the particular therapeutic effect to be achieved.

Toxicity and therapeutic efficacy of the compositions described herein can be determined by standard pharmaceutical procedures in cell cultures or experimental animals, e.g., by determining the IC₅₀ (the concentration which provides 50% inhibition), LD₅₀ (the dose lethal to 50% of the population) and the ED₅₀ (the dose therapeutically effective in 50% of the population) and the maximal tolerated dose for a subject compound. The dose ratio between toxic and therapeutic effects is the therapeutic index and it can be expressed as the ratio LD₅₀/ED₅₀. Antibodies that exhibit large therapeutic indices may be less toxic and/or more therapeutically effective.

The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, and all other relevant factors.

Therapeutic Use

CCL20 neutralizing or antagonizing agents according to the invention are used, in accordance with a currently preferred embodiment of the invention, for the treatment of cancer. It is herein reported for the first time that a CCL20-neutralizing antibody in accordance with the invention elicits anti-tumor effects in a variety of tumors. Within the scope of the present invention, methods are provided for the use of the anti-CCL20 antibody for the treatment of a tumor by administering to a subject an effective amount of the antibody of the invention.

The antibodies or compositions of the invention are administered in therapeutically effective amounts as described. Therapeutically effective amounts may vary with the subject's age, condition, sex, and severity of medical condition. Appropriate dosage may be determined by a physician based on clinical indications. The antibodies or compositions may be given as a bolus dose to maximize the circulating levels of antibodies for the greatest length of time. Continuous infusion may also be used after the bolus dose.

In the context of cancer therapy, a “therapeutically effective amount” of an anti-CCL20 antibody refers to an amount of an antibody which is effective, upon single or multiple dose administration to a subject (such as a human patient) at treating, preventing, curing, delaying, reducing the severity of, and/or ameliorating at least one symptom of cancer, or prolonging the survival of the subject beyond that expected in the absence of such treatment.

The effective amount required to achieve the therapeutic end result may depend on a number of factors including, for example, the tumor type and the severity of the patient's condition (i.e. the cancerous state), and whether the antibody is co-administered together with another agent which acts together with the antibody in an additive or synergistic manner. The antibody may be administered e.g. following detection of primary or secondary tumors in the subject.

As used herein, the term “subject” is intended to include human and non-human animals. Subjects may include a human patient having a disorder, in which cells that express CCR6 and/or CXCR4, e.g. cancer cells, contribute to the etiology or pathology of the disorder.

Examples of dosage ranges that can be administered to a subject can be chosen from: 1 μg/kg to 20 mg/kg, 10 μg/kg to 2 mg/kg, 100 μg/kg to 10 mg/kg, 500 μg/kg to 2 mg/kg, 1 mg/kg to 10 mg/kg, 1 mg/kg to 5 mg/kg and 5 mg/kg to 10 mg/kg (or higher). These dosages may be administered daily, weekly, biweekly, monthly, or less frequently, for example, biannually, depending on dosage, method of administration, disorder or symptom(s) to be treated, and individual subject characteristics. Dosages can also be administered via continuous infusion (such as through a pump). The administered dose may also depend on the route of administration. For example, subcutaneous administration may require a higher dosage than intravenous administration. Typically, a dose of 1-10 mg/kg is administered (by injection or infusion) daily or twice a week to human cancer patients.

Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration of the antibodies by modifications such as, for example, lipidation.

In a preferred aspect, the antibody is substantially purified (e.g., substantially free from substances that limit its effect or produce undesired side-effects).

The administration of said compositions can be typically achieved by means of parenteral administration, e.g., intravenously (i.v.) intraperitoneally (i.p.) or intramuscularly (i.m.). Methods of treatment may comprise pharmaceutical compositions of the antibodies according to the invention. Other delivery systems are known and can be used to administer an antibody of the present invention, including e.g. encapsulation in liposomes, microparticles, microcapsules or receptor-mediated endocytosis. Alternatively or additionally, methods of treatment may include gene therapy (e.g. construction of a nucleic acid as part of a retroviral or other vector as known in the art) and cell therapy, ex-vivo or in-vivo wherein cells are autologous or allogeneic.

Methods of introduction include but are not limited to intradermal, intramuscular, intraperitoneal, intravenous, subcutaneous, intranasal, epidural, inhalation and oral routes. The antibodies or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. Pulmonary administration can also be employed, e.g., by use of an inhaler or nebulizer, and formulation with a porous, or gelatinous material, including membranes, such as sialastic membranes, or fibers. Typically, when administering an antibody of the invention, care must be taken to use materials to which the protein does not absorb.

In another embodiment, the invention provides a method for treating a CCL20 dependent cancer in a subject in need thereof, comprising administering to the subject a therapeutically effective amount of an agent that specifically binds and neutralizes CCL20, and optionally at least one pharmaceutically acceptable excipient.

In another embodiment, the cancer is a CCR6 expressing cancer. In another embodiment, the cancer is a CXCR4 expressing cancer. In another embodiment, the cancer expresses both CCR6 and CXCR4.

As used herein, a CXCR4- or CCR6-expressing cancer refers to a neoplastic disorder in which the subject is afflicted with a tumor characterized by surface expression of CXCR4 or CCR6 in at least a part of the cells of a tumor. Such tumors are disclosed herein as CCL20 dependent tumors, i.e. tumors in which CCL20 is known to facilitate tumor initiation, growth, progression and/or spreading.

For example, the cancer may be selected from gliomas, leukemia, uterine cancer, lymphoma (e.g. Burkitt's lymphoma), neuroblastomas, pancreatic cancer (e.g. pancreatic adenocarcinomas), prostate cancer (e.g. carcinomas), clear cell renal carcinoma, colorectal, lung, and breast tumors (e.g. adenocarcinomas) and melanoma, wherein each possibility represents a separate embodiment of the present invention. In a particular embodiment, the cancer is other than pancreatic cancer. In another particular embodiment the cancer is other than hepatocellular carcinoma. In yet another particular embodiment, the cancer is prostate cancer (e.g. a CCL20-dependent prostate carcinoma). In a further particular embodiment, the cancer is colon cancer (e.g. a CCL20-dependent colorectal carcinoma).

As used herein, “treating” cancer (or treating a subject with cancer) refers to taking steps to obtain beneficial or desired results, including but not limited to, alleviation or amelioration of one or more symptoms of cancer, diminishment of extent of disease, delay or slowing of disease progression, amelioration, palliation or stabilization of the disease state, partial or complete remission, prolonged survival and other beneficial results known in the art.

In another embodiment, the compositions of the invention are useful for inhibiting, preventing or reducing metastasis in a subject in need thereof, wherein the metastasizing cells are CCL20 dependent.

As used herein, the term “inhibiting” or “reducing” refer to either statistically significant inhibition or reduction, or to inhibition or reduction to a significant extent as determined by a skilled artisan, e.g. the treating physician. It should be understood, that inhibition or reduction does not necessarily indicate a total elimination of the measured function or biological activity. A reduction in activity may be for example about 10%, 20%, 30%, 40%, 50%, 60%, 70%, 80%, 90%, or more.

In another embodiment, the invention is directed to the use of a CCL20-neutralizing antibody for the preparation of a medicament. In one embodiment, the medicament is identified for the treatment of cancer. In another embodiment, the cancer is a CCL20 dependent cancer. In another embodiment, the cancer is a CCR6 expressing cancer (i.e. expresses CCR6 on the surface of at least a portion of the cancer cells). In another embodiment, the cancer is a CXCR4 expressing cancer. In another embodiment, the cancer expresses both CCR6 and CXCR4. For example, the cancer may be selected from glioma, leukemia, uterine cancer, lymphoma (e.g. Burkitt's lymphoma), neuroblastomas, pancreatic cancer (e.g. pancreatic adenocarcinomas), prostate cancer (e.g. carcinomas), clear cell renal carcinoma, colorectal, lung, and breast tumors (e.g. adenocarcinomas) and melanoma. In a particular embodiment, the cancer is prostate cancer. In another particular embodiment, the cancer is colon cancer. In various other embodiments, the medicament is useful for inhibiting or reducing tumor progression, growth or vascularization, for reducing the size of an existing tumor and/or for inhibiting or preventing tumor invasiveness or metastasis.

The following examples are presented in order to more fully illustrate some embodiments of the invention. They should, in no way be construed, however, as limiting the broad scope of the invention.

### EXAMPLES

### Experimental Procedures

Cell Culture

The following human cell lines were used in the study: prostate cell lines PC3 (CRL-1435), LNCaP (CRL-10995), 22Rv1 (CRL-2505), DU145 (HTB-81); acute promyelocytic leukemia cell lines NB4, HL-60 (CCL-240) and colon carcinoma cell line HT-29 (HTB-38). All cell lines were purchased from ATCC and were maintained at log growth in RPMI medium (Biological Industries, Kibbutz Beth Haemek, Israel) supplemented with 10% fetal calf serum (FCS), 1 mM L-glutamine, 100 U/ml penicillin, and 0.01 mg/ml streptomycin (Biological Industries) in a humidified atmosphere of 5% CO₂ at 37° C.

Transduction of Cell Lines and Single-Cell Clone Production

Generation of stably CCL20-overexpressing PC3 single-cell clones: the construct pcDNA3-CCL20 was generated by isolation of CCL20 fragment from pORF hMIP3a vector (InvivoGen Delivering Genes, San Diego, Calif., USA) and insertion into pcDNA3 (Invitrogen, Carlsbad, Calif., USA) in XbaI and EcoRV sites. PC3 cell line stably overexpressing CCL20 (PC3-CCL20) was produced by transfection of PC3 cells with 2 μg of pcDNA3-CCL20 construct using TransIT-LT1 Minis reagent (Gene Transfer, Madison, Wis., USA) according to manufacturer's instructions. Transfected cells were grown in selection medium containing G418 1 mg/mL. PC3-CCL20 single-cell clones were produced by limited dilutions. The level of secreted CCL20 protein was analyzed in supernatant of PC3-CCL20 clones using commercially available ELISA kit (R&D Systems, Minneapolis, Minn.).

Generation of stably CXCR4-overexpressing PC3 single-cell clones: PC3 cell line overexpressing the luc and the bicistronic CXCR4-GFP genes (PC3-CXCR4) was generated in our laboratory as previously described (Darash-Yahana et al., 2004). PC3-CXCR4 single-cell clones were produced by limited dilutions. The level of surface CXCR4 expression was determined using flow cytometric (FACS) analysis.

Immunohistochemistry and Scoring

Two different commercial prostate tumor tissue microarrays were used: CA2 array included 52 prostate cancer tissue sections and CA3 array included 48 prostate cancer tissue sections (SuperBioChip Lab).

In addition tissue samples of primary prostate tumors from 41 patients, 2 were collected from the archives of the Pathology Department of the Hadassah Medical Organization, Jerusalem, Israel. Formalin-fixed, paraffin-embedded tissue samples were initially dewaxed, rehydrated, treated with EDTA buffer and blocked with CAS blocking reagent (Zymed Laboratories, San Francisco, Calif., USA) for 30 minutes in room temperature. Samples were then incubated overnight at 40C in a humidified chamber with anti-human CCL20 polyclonal antibody (PeproTech EC, London, UK), diluted to final concentration 20 μg/ml, or alternatively with anti-human CCR6 monoclonal antibody (R&D Systems, Minneapolis, Minn.) diluted to final concentration 10 μg/ml, or with anti-human CXCR4 monoclonal antibody, clone 12G5 (R&D Systems, Minneapolis, Minn.) diluted to final concentration 10 μg/ml. Next, the sections stained for CCL20 were incubated with diluted 1:1000 biotinylated goat-anti-rabbit antibody (Jackson ImmunoResearch), for 30 minutes at room temperature and thereafter with horseradish peroxidase-conjugated streptavidin (Zymed Laboratories, San Francisco, Calif., USA) for 30 minutes at room temperature. The sections stained for CCR6 or CXCR4 were incubated with secondary anti-mouse horseradish peroxidase-conjugated antibody (DakoCytomation, Glostrup, Denmark) for 30 minutes at room temperature. 3-amino-9-ethylcarbazole (AEC) was used for color development, and sections were counterstained with hematoxylin.

Analysis of CCL20 and CCR6 expression was determined by scoring the staining intensity as negative, weak or strong by two independent investigators. Scoring was performed blindly, without knowledge of overall Gleason score or tumor pathologic stage.

RNA Extraction and Reverse Transcription

Total RNA was extracted from prostate and leukemic cell lines using TRIzol reagent (Invitrogen Life Technologies) according to the protocol recommended by manufacture. For cDNA synthesis, 2.5 microgram of total RNA were reverse-transcribed in a final reaction volume of 25 μL containing 1× M-MLV RT buffer, 2.5 μmol/L random hexamers, 0.5 mmol/L each dNTP, 3 mmol/L MgCl2, 0.4 U/μL RNase inhibitor, and 100 U/μL M-MLV RT. All RT reagents were purchased from Promega, Madison, Wis. The reaction conditions were 1 min at 90° C., 1.5 hour at 42° C., and 15 min at 75° C.

Semi-Quantitative PCR

The following primer pairs were used for PCR: β-actin sense 5′-CCCTGGACTTCGAGCAAGAG′-3′ (SEQ ID NO: 2), antisense 5′-TCTCCTTCTGCATCCTGTCG-3′ (SEQ ID NO: 3); CCL20 sense 5′-ATGTGCTGTACCAAGAGTTT-3′ (SEQ ID NO: 4), antisense 5′-CAAGTCTGTTTTGGATTTGC-3′ (SEQ ID NO: 5); CCR6 sense 5′-CCATTCTGGGCAGTGAGTCA-3′ (SEQ ID NO: 6), antisense 5′-AGCAGCATCCCGCAGTTAA-3′ (SEQ ID NO: 7); CXCR4 sense 5′-AGCTGTTGGCTGAAAAGGTGGTCTATG-3′ (SEQ ID NO: 8), antisense 5′-GCGCTTCTGGTGGCCCTTGGAGTGTG-3′ (SEQ ID NO: 9); CXCL12 sense 5′-ATGAACGCCAAGGTCGTGGTCG-3′ (SEQ ID NO: 10), antisense 5′-TGTTGTTGTTCTTCAGCCG-3′ (SEQ ID NO: 11). Two microliters of the reverse-transcribed product were subjected to PCR amplification in a final reaction volume of 20 containing 1 U of Supertherm Taq polymerase (JMR-Holdings, London, England). Amplification conditions were denaturation at 94° C. for 30 seconds, annealing at 56° C. for 30 seconds, and extension at 72° C. for 30 seconds for 30 consecutive cycles. The PCR amplified products were run on 1% agarose gel containing ethidium bromide. The sizes were estimated by comparison with molecular weight markers.

Real-Time PCR

CCL20 quantitative PCR assay containing the primers and probe mix was purchased from Applied Biosystems, Foster City, Calif., and utilized according to the manufacturer's instructions. PCR reactions were carried out in a final reaction volume of 20 μL containing 100 ng cDNA template, 10 μL TaqMan Universal Master Mix (Applied Biosystems), and 1 μL gene and probe mix. All reactions were run in triplicates using ABI Prism 7700 Sequence Detector System (Applied Biosystems). For each RNA sample threshold cycle numbers (Ct) were determined using Sequence Detector Software (version 1.6; Applied Biosystems) and transformed using the ΔCt method as described by the manufacturer. Gene expression of CCL20 gene was analyzed in relation to the levels of the housekeeping β-actin gene.

Cell Proliferation Assay

The effect of CCL20 on the viability of PC3, PC3-CXCR4.5, PC3-CCL20 clones and leukemia NB4 and HL60 cells was studied. In brief, PC3 and PC3-CXCR4.5 cells were seeded at 2×10⁴ cells/1 ml per well into a 24-well plate in medium supplemented with 0.1% FCS with or without various concentrations of CCL20 (PeproTech EC, London, UK). The cells were incubated for six days. Following three days the medium with or without CCL20 was renewed. On day six, the attached cells were harvested, stained with propidium iodide (Sigma, St. Louis, Mo.), and the number of viable cells was determined using FACS analysis.

Optionally, PC3 and PC3-CXCR4.5 cells were labeled with 5-bromo-2-deoxyuridine (BrdU) (Sigma, St. Louis, Mo.) at concentration of 10 μM during the last 16 hours of incubation and processed for BrdU detection using specific anti-BrdU antibody (eBioscience) and FACS analysis.

Cell Adhesion Assay

Prostate cancer cells (PC3, PC3-CXCR4.5 and PC3-CCL20 clones), leukemia NB4 and HL60 cells, and colon cancer cells HT-29 (1×10⁵/500 μl) were allowed to adhere to 10 μg/ml fibronectin-coated or collagen type I-coated 24-well plates for 30 minutes at 37° C. in serum-free RPMI supplemented with 0.1% bovine serum albumin (BSA). Non-adherent cells were washed twice with cold PBS. Adherent cells were collected in 300 μl FACS buffer (PBS×1+0.1% BSA+0.01% NaNO₃) with 5 mM EDTA and counted by FACScalibur (Becton Dickinson Immunocytometry Systems).

ELISA Assay

Prostate cancer cells (PC3, PC3-CXCR4.5 and PC3-CCL20 clones), leukemia cells NB4 and HL60 and colon cancer cells HT-29 were seeded into a 12-well plate at 2×10⁵/1 ml of medium per well with various concentrations of CXCL12 (5-1000 ng/ml) (PeproTech EC, London, UK) or PTX (List Biological Laboratories, Campbell, Calif., USA). The cells were incubated for 48 hours, supernatants were collected and CCL20 protein levels were determined using sandwich-type ELISA commercially available kit according to the manufacture's protocol (R&D Systems, Minneapolis, Minn.). The absorbance was read at 450 nm.

Flow Cytometric Analysis

In order to characterize the expression levels of chemokine receptors CXCR4 and CCR6 and adhesion molecules VLA1, VLA2, VLA4, VLA5, LFA1 and L-selectin on cancer cell lines, the cells were stained with human specific direct-labeled antibodies and analyzed by FACScalibur (Becton Dickinson Immunocytometry Systems), using CellQuest software. For CXCR4 expression analysis anti-human CXCR4 monoclonal antibody, clone 12G5 (R&D Systems, Minneapolis, Minn.) or polyclonal anti-N-terminus antibody (Chemicon International, Temecula, Calif., USA) were used. For CCR6, anti-human CCR6 monoclonal antibody, clone 53103.11 (R&D Systems, Minneapolis, Minn.) was used. Antibodies for VLA2, VLA4, VLA5 and L-selectin were purchased from R&D Systems, Minneapolis, Minn. Antibodies for VLA1 were purchased from Chemicon International, Temecula, Calif., and antibodies for LFA-1—from IQ Products, Groningen, Netherlands. Primary antibodies and matched isotype controls were purchased from the same companies,

Establishment of Tumor Xenografts

SCID/beige mice (C.B-17/IcrHsd-SCID-bg) were maintained under defined flora conditions at the Hebrew University Pathogen-Free Animal Facility. All experiments were approved by the Animal Care Committee of the Hebrew University. Prostate cancer cell lines (PC3, PC3-CXCR4.5, PC3-CCL20.30 and PC3-CCL20.10) were grown to 80% confluence, harvested, resuspended in 1× phosphate-buffered saline and were injected subcutaneously in the flank of male SCID/beige mice (5×10⁶/mouse). Once palpable, tumors were measured using vernier caliper, and tumor size (width×length) was calculated. For the neutralizing experiments mice were treated with subcutaneous injections of monoclonal anti-human CCL20 antibody (MAB360, R&D Systems, Minneapolis, Minn.) or control IgG1 antibody, three times a week, 20 μg/mouse. At the end of the experiments animals were sacrificed, tumors were harvested, measured and weighted.

MRI Analysis of Tumor Growth and Blood Vessel Functionality and Maturation

MRI experiments were performed on a horizontal 4.7 T Bruker Biospec spectrometer, using a birdcage coil. Nine mice from PC3-CCL20.30 group and 5 from control PC3LG group were anesthetized (pentobarbital, 30 mg/kg IP) and placed supine at the center of the coil. For the analysis of tumor size, coronal and axial T2 weighted fast spin echo images (TR/TE=2000/37 ms) were acquired. Functionality and maturation of the neovasculature were determined from T2*-weighted gradient echo (GE) images (TR/TE=147/10 ms; flip angle=30; field of view=5 cm; 256×128 pixels; 5 slices with slice thickness=0.6 mm; 2 averages) acquired during breathing of air, air-CO₂ (95% air and 5% CO₂), and carbogen (95% oxygen and 5% CO₂) as previously described. Eight repeats were acquired at each gas mixture.

MRI Data Analysis

MRI data were analyzed using the IDL software (Research Systems, Inc.). Maps of mean SI values for each pixel during the different inhaled gases (Sair, Sco2 and So2) were calculated from four repeats for each gas. The percentage of change of fMRI SI induced by hypercapnia (ΔSco2) and hyperoxia (ΔSo2) was calculated (for each pixel≧noise threshold) using the following equations:

$$Δ {S}_{{CO}_{2}} = \frac{{\overline{S}}_{{CO}_{2}} - {\overline{S}}_{air}}{{\overline{S}}_{air}} \times 100 Δ {S}_{{O}_{2}} = \frac{{\overline{S}}_{{O}_{2}} - {\overline{S}}_{{CO}_{2}}}{{\overline{S}}_{{CO}_{2}}} \times 100$$

The mean±SD values of ΔSo2 and ΔSco2 were calculated from a region of interest containing the whole tumor, and normalized to the contra-lateral muscle. ΔSo2 measures the capacity of erythrocytes to deliver oxygen from the lungs to each pixel in the image thus reflecting vessel density and functionality (ref). ΔSco2 corresponds to vessel maturation since only mature vessels, coated with smooth muscle cells, will react to CO₂.

Statistical Analysis

Data are presented as means±SD or ±SE. Statistical comparison of means was performed by a two-tailed unpaired Student's t test. Differences with a P<0.05 were determined as statistically significant. Statistical analysis of the Immunohistochemical staining was performed using two-tailed Mann-Whitney test.

### Example 1

### CCL20 Promotes the Growth and Adhesion of CCR6-Expressing Tumor Cells In Vitro

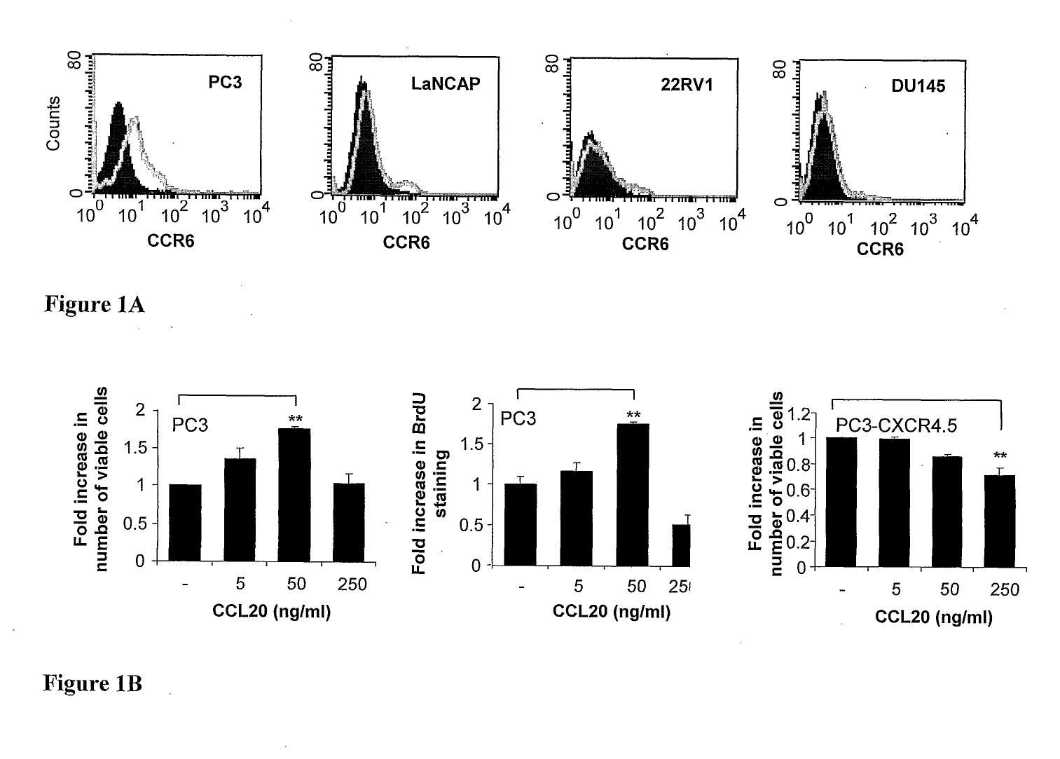

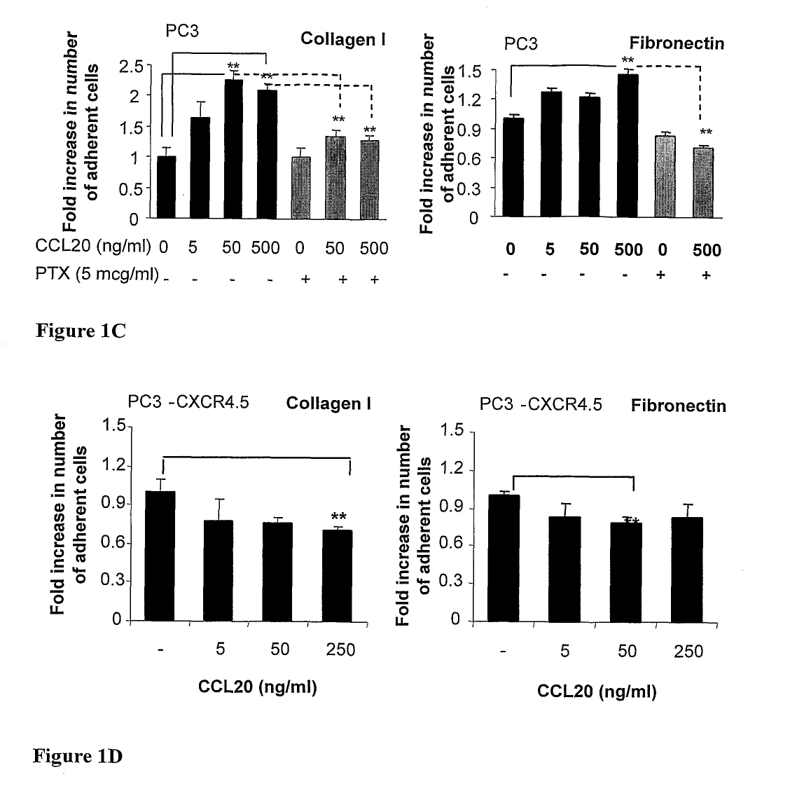

To investigate the role of CCL20 in prostate cancer development, the expression of CCL20 and its receptor CCR6 was characterized in human prostate cell lines PC3, LNCaP, 22RV1 and DU145. First, CCR6 receptor surface and mRNA expression levels was examined in these four cell lines. RT-PCR analysis and FACS analysis demonstrated that only PC3 cell line expressed CCR6 receptor on mRNA level and on the cell surface (FIG. 1A). Next, ELISA experiments were performed to determine the secretion levels of CCL20 chemokine. Among the four prostate cancer cell lines studied only PC3 cells secreted detectable levels of CCL20 into the culture supernatant during the 48 hours incubation. However, in addition to PC3 cells, the mRNA expression of CCL20 was demonstrable in DU145 cells and at a very low level also in LANCaP cells.

To assess biological behavior resulting from CCL20-mediated activation in PC3 cells, which co-express the CCR6 receptor and its ligand CCL20, the effect of CCL20 on the growth and survival of PC3 cells in culture was studied. PC3 cells were treated with various concentrations of CCL20. The number of viable cells following six days of incubation was detected using propidium iodide (PI) and FACS analysis. Treatment of cells with CCL20 increased the number of viable PC3 cells in the culture at a concentration of 5 ng/ml (1.35-fold increase, p<0.04) and at a concentration of 50 ng/ml (1.75-fold increase, p<0.0004). At the highest concentration of CCL20, 250 ng/ml, no change the number of viable cells compared to control non-stimulated PC3 cells was observed (FIG. 1B). To further verify these results, CCL20-treated cells were loaded with 5-bromo-2-deoxyuridine (BrdU). Cell proliferation was tested by staining for BrdU incorporation using specific anti-BrdU antibody and FACS analysis. Consistent with previous results, CCL20 induced the incorporation of BrdU to the cells at a concentration of 5 and 50 ng/ml (1.76-fold increase, p<0.0002), whereas treating the cells with higher concentration of CCL20 did not change the level of BrdU incorporation (FIG. 1B). In accordance with the previous results, PC3-CXCR4.5 cells, which constitutively express high levels of CCL20, were inhibited in their growth when treated with increasing concentrations of CCL20 (FIG. 1B). Concentration of 250 ng/ml even decreased the number of PC3-CXCR4.5 viable cells (p<0.006).

Adhesion of cancer cells to extracellular matrix (ECM) components is a step that is associated with tumor seeding, invasion and spreading. Since integrins are critically involved on cell to ECM adhesion, the surface expression of the integrins VLA-1, VLA-2, VLA-4, VLA-5 and LFA-1 on PC3 and PC-CXCR4.5 cells was first analyzed by flow cytometry. Analysis of integrin subunits cell-surface expression showed that PC3 and PC3-CXCR4.5 cells express high levels of collagen receptor VLA-2 (α2) subunit, without significant difference in expression between PC3 and PC3-CXCR4.5 cells. In addition, the cells express less abundant level of collagen receptor VLA-1 (α1) subunit. The fibronectin receptor VLA-5 (α5) subunit was expressed at low level on PC3 and PC3-CXCR4.5 cells. In contrast, the VCAM-1 and Fibronectin receptor VLA-4 (α4) and the ICAM-1/2/3 receptor LFA-1 were not detected on the cell surface of both cell lines. In order to investigate the effect of CCL20 on PC3 cell adhesion to the ECM proteins, the adhesion of PC3 cells to the fibronectin and collagen I was tested in response to increasing concentration of CCL20. As shown in FIG. 1C, elevated doses of CCL20 slightly increased the adhesion of cells to fibronectin (500 ng/ml of CCL20 promoted 1.45-fold increase, p<0.01), and significantly increased the adhesion of cells to collagen type I in a dose-dependent manner (FIG. 1C). Upon activation by CCL20 at concentration of 50 ng/ml PC3 adhesion to collagen I was 2.2-fold elevated (p<0.001), concentration of 500 ng/ml caused 2.09-fold increase in adhesion (p<0.003). Treatment with pertussis toxin (PTX) prevented the CCL20-induced increase in PC3 cell adhesion to fibronectin (p<0.0009) and collagen I (p<0.002) (FIG. 1C). In contrast to PC3 cells, adhesion of CCL20-producing PC3-CXCR4.5 cells to collagen I and fibronectin was slightly decreased following stimulation with increased doses of CCL20 (FIG. 1D).

In addition to PC3 cells, the effect of CCL20 activation on biological function of other human prostate cancer cell lines—LNCaP, 22RV1 and DU145 cells was tested. In vitro proliferation and adhesion of the cells to collagen were tested in the absence or presence of different concentrations of CCL20. In agreement with CCR6 expression pattern, LNCaP, 22RV1 and DU145 cells that do not express CCR6, did not respond to CCL20 stimulation and no increase in proliferation or adhesion was observed.

Thus, CCL20 can stimulate both PC3 cell proliferation and adhesion to collagen type I in a dose dependent manner.

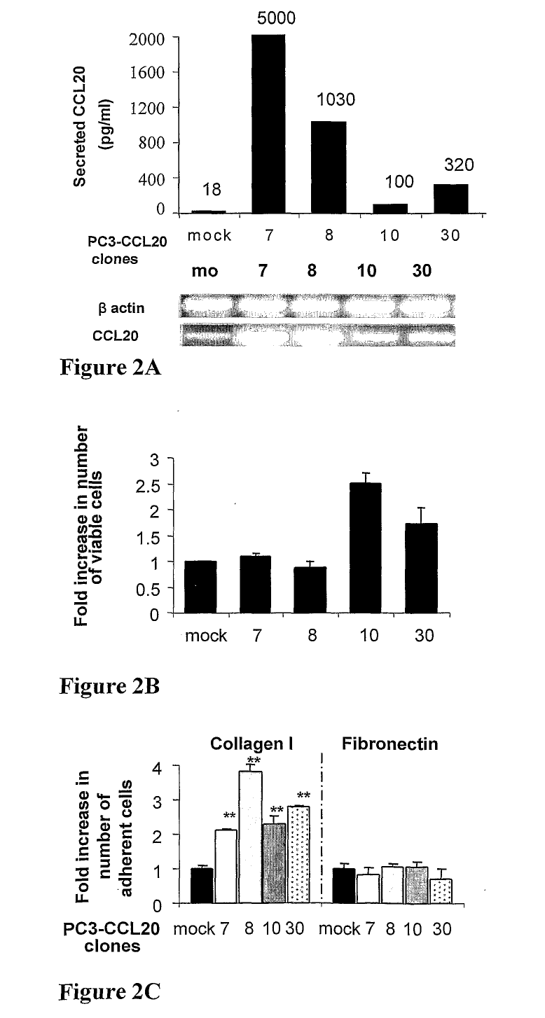

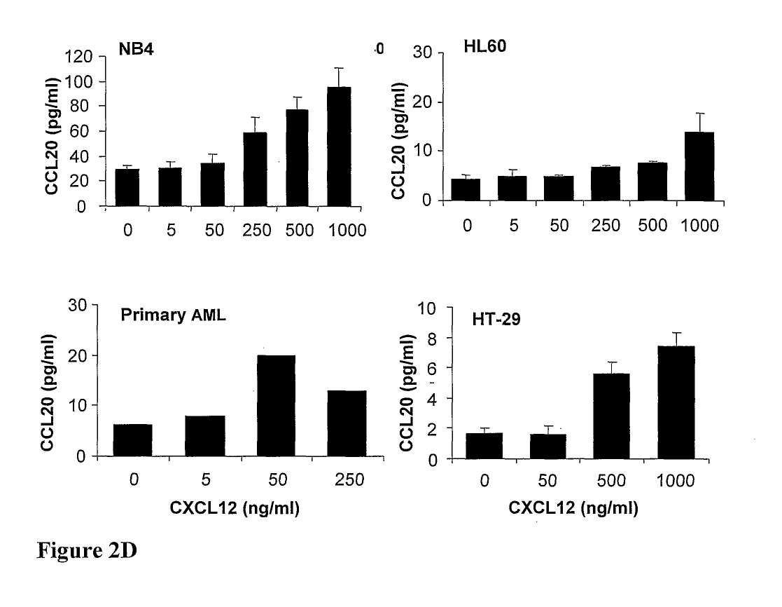

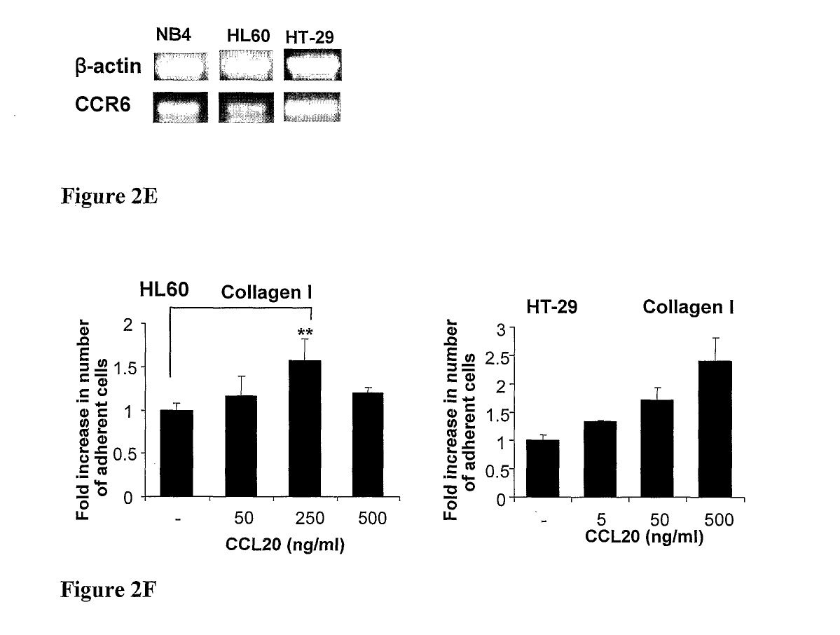

In order to further determine the involvement of CCL20 in cancer development in vitro, the CCL20 gene was introduced into prostate PC3 cells which express the CCR6 receptor. PC3 cells were stably transfected with vector encoding CCL20, and different clones overexpressing CCL20 were obtained. The levels of CCL20 were quantified using PCR and ELISA assay (FIG. 2A). Clones number 7 and 8 demonstrated the highest levels of CCL20 secretion (5000 and 1030 pg/ml, respectively) whereas clones 10 and 30 showed moderate levels of CCL20 secretion (100 and 320 pg/ml, respectively).

Next, the growth of PC3 clones that over express CCL20 was tested. The number of viable cells following six days of incubation was detected using PI and FACS analysis. PC3-CCL20 clones that secrete moderate levels of CCL20, PC3-CCL20.10 and PC3-CCL20.30, demonstrated 1.7-fold and 2.5-fold increase in number of viable cells in culture, respectively (FIG. 2B). The growth of PC3-CCL20 clones that secrete high levels of CCL20 (PC3-CCL20.7 and PC3-CCL20.8) was similar to parental control PC3 cells (FIG. 2B). This may suggest that autocrine secretion of CCL20 may drive tumor growth.

To further explore the effect of CCL20 overexpression on prostate cancer cell behavior, the adhesion of PC3 clones that over express CCL20 to ECM proteins fibronectin and collagen I was assessed. CCL20-overexpressing PC3 cells were grown to confluence, harvested, and allowed to adhere to fibronectin or collagen I-coated plates. Over-expression of CCL20 significantly increased the adhesion of all four CCL20-expressing clones to the collagen I. Comparing to mock-transfected PC3 cells, clones 7, 8, 10 and 30 demonstrated 2.1-fold (p<0.04), 3.8-fold (p<0.01), 2.2-fold (p<0.03) and 2.8-fold (p<0.004) increase their adhesion to collagen I, respectively (FIG. 2C). No significant change observed in adhesion of these cells to the fibronectin.

Next, ELISA was used to examine the expression of CCL20 in a range of human cancer cell lines. It was found that in addition to PC3 cells, CCL20 is secreted by promyelocytic leukemia (APL) cell lines, NB4 and HL60, by primary blasts of patient with acute myelocytic leukemia as well as by human HT-29 colon carcinoma cells (FIG. 2D). In NB4, HL60, primary AML blasts and in HT-29 cells, the secretion of CCL20 was increased following stimulation with CXCL12, in a dose-dependent manner (FIG. 2D). Next, the expression of the CXCR4 receptor on NB4, HL60 cells, AML blasts and HT-29 cells was characterized. Leukemic lines NB4 and HL60, primary human AML blasts and HT-29 cells demonstrated high cell-surface expression levels of CXCR4 receptor. These results suggest a more general role for CXCR4 in regulating CCL20 expression in various cancer cells.

To confirm the role of CCL20 in autocrine stimulation of cancer cells from different origins, the expression of CCR6 in CCL20-secreting NB4, HL60, and HT-29 cells was tested. It was found that NB4, HL60, and HT-29 cells expressed CCR6 on mRNA level (FIG. 2E), however HL60 possessed higher levels of cell-surface CCR6 then NB4 and HT-29 cells. Next, the effect of CCL20 stimulation on NB4, HL60, and HT-29 cell proliferation and adhesion to ECM components collagen I and fibronectin was tested. A 1.6-fold increase in adhesion of HL60 cells to collagen I was found upon simulation with CCL20 250 ng/ml (p<0.006) and 2-fold increase in adhesion to fibronectin was found upon stimulation with 500 ng/ml (p<0.015) (FIG. 2F). Stimulation of HT-29 resulted in a dose dependent increase adhesion to collagen type I but not fibronectin (FIG. 2F). In contrast to HL60 and HT-29, NB4 cells that express low surface level of CCR6 did not proliferate or adhere to fibronectin or collagen in response to CCL20.

### Example 2

### Overexpression of CCL20 Increases the Growth, Invasion and Vascularization of PC3 Prostate Cells In Vivo

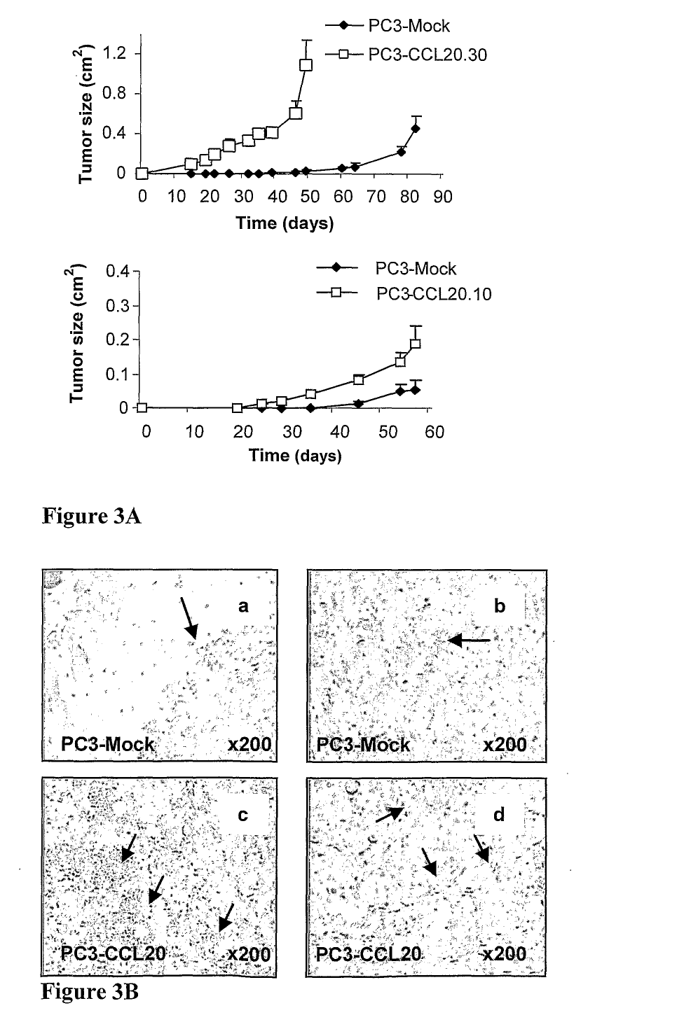

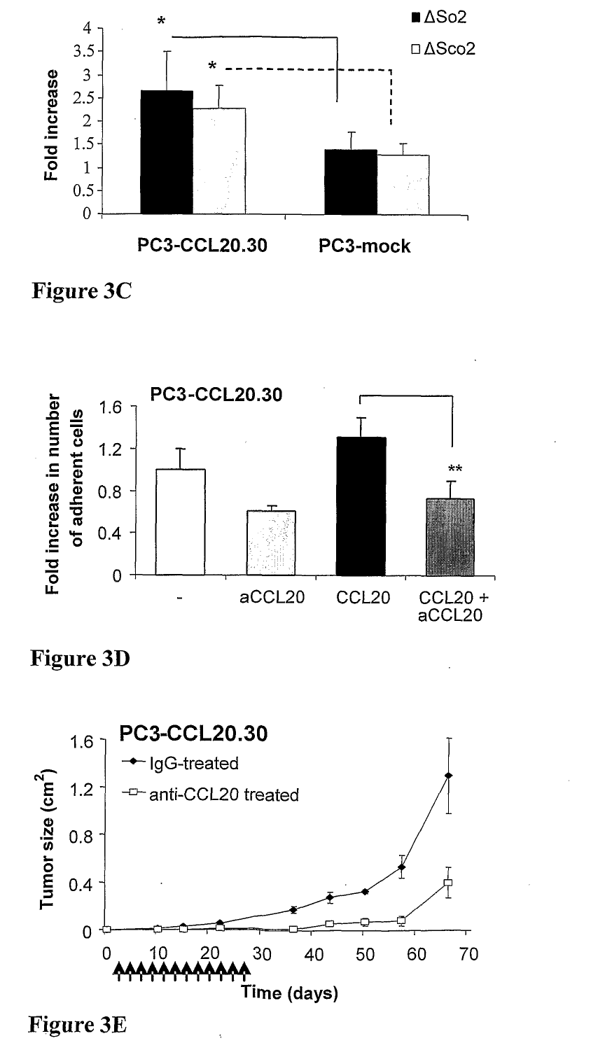

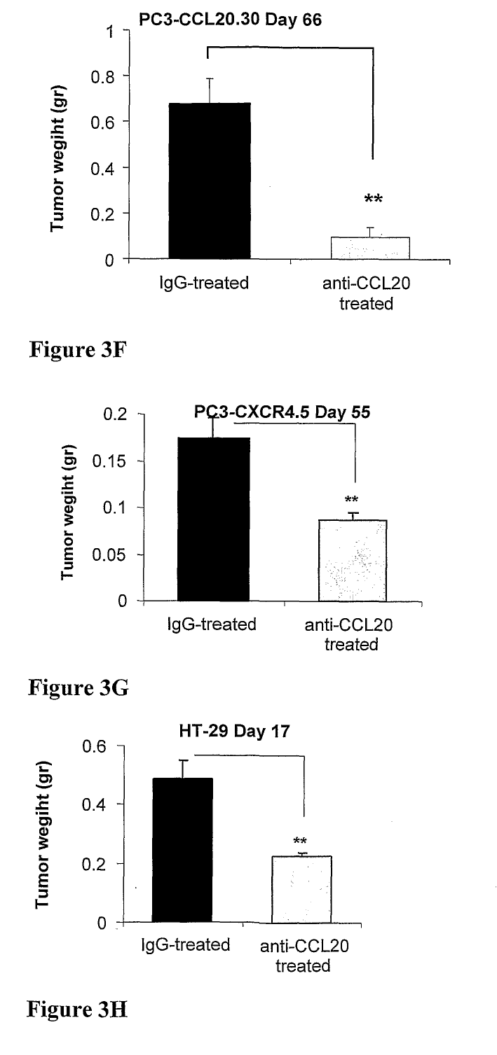

To determine the role of CCL20 in tumor development in vivo, a tumor xenograft model was used. Human mock-transfected and CCL20-overexpressing PC3 cells were injected subcutaneously into SCID/bg mice. For in vivo experiments PC3-CCL20 clones 10 and 30 were chosen, since these clones demonstrated increased proliferation rate in culture, and produced either comparable levels of CCL20 (100, and 320 pg/ml) to PC3-CXCR4.5. Mice injected with PC3-CCL20.30 cells developed larger tumors as measured by increase in size comparing to mice injected with mock-transfected PC3 cells (FIG. 3A). Moreover, tumors produced by PC3-CCL20.30 cells were more vascularized and invasive to the neighboring tissues (muscle and dermis) (FIG. 3B). These findings were confirmed by H&E-stained tissue sections of xenograft tumors. Histological analysis of PC-CCL20 tumors demonstrated the invasion of tumors to adjacent muscle tissue, necrosis that can be associated with rapid tumor growth, and massive aberrant vascularization of tumors. In contrast, PC3-mock tumors were encapsulated, non-invasive and no abberant blood vessels were present (FIG. 3B). Mice injected with PC3-CCL20.10 cells also developed larger tumors as measured by increase in size and weight comparing to mice injected with mock-transfected PC3 cells. However, the difference between PC3-CCL20.10 and the parental cells were smaller (FIG. 3A).

Both PC3-CCL20.30 and the parental PC3 cells developed a necrotic core, while tumors that overexpress CCL20 continue to grow beyond this point, suggesting that CCL20 may facilitate angiogenesis in tumors. This hypothesis was studied by comparing the number of blood vessels in histological sections as well as by using an in vivo intra-tumoral vessel functionality MRI based assay in the CCL20 tumors (PC3-CCL20.30) versus that in control tumors (PC3-mock). Macroscopic assessment of tumors overexpressing CCL20 revealed increased vascularization as compared to control tumors (FIG. 34C). To complement the data obtained from histology on blood vessel density, vessel functionality (ΔSo2) was measured by fMRI to study the actual in vivo perfusion of the tumor and the oxygen delivery efficiency into the tumor mass. Functionality of the vasculature was derived from GE images acquired during inhalation of air-CO₂ and carbogen (95% oxygen+5% CO₂) (Barash et al., 2007) in mice implanted with PC3-mock cells or with PC3-CCL20.30 cells. Interestingly, MRI analysis showed that ΔSo2 values from PC3-CCL20.30 tumors were significantly higher (FIG. 3C). ΔSo2 maps derived on day 41 showed enhanced tumor vascularity in tumors produced by PC3-CCL20.30 vs. control tumors that had very low ΔSo2 values. The control tumors were left to grow for an additional month in order to let them reach a similar size. However, even on day 71 they were significantly less vascularized. While functional vessels were observed at the center of tumors overexpressing CCL20, no functional vessels were observed at the center of the control tumors only on the borders of these tumors. The mean±SD values of ΔSo2 were calculated from a region of interest containing the whole tumor, and normalized to contra-lateral muscle, pooling data from 9 mice from the PC3-CCL20.30 group and 5 mice from the PC3-mock group (four slices/mouse; FIG. 3C, p<0.001). These results suggest that high expression of CCL20 results in early neovascularization of the tumors while in control tumors the development of necrosis was mediated by poor perfusion. These results suggest an involvement of CCL20 in prostate tumor growth, spreading, invasiveness and vascularization.

### Example 3

### Neutralization of CCL20 Inhibits the CCL20 and CXCR4-Dependent Growth of Prostate Tumors

Having established the role of CCL20 in cancer development in vivo, the effect of neutralizing antibodies to human CCL20 on the growth of CCL20-and CXCR4 overexpressing PC3 cells was evaluated. First, the ability of anti-CCL20 antibodies to neutralize the CCL20-induced adhesion in vitro of PC3-CCL20.30 cells to collagen I was tested. The presence of monoclonal anti-human CCL20 antibodies abolished the adhesion of PC3-CCL20.30 cells to collagen I in response to CCL20 stimulation (FIG. 3D).

Next, the in vivo potential of neutralizing anti-CCL20 antibodies was assessed. PC3-CCL20.30 cells were injected subcutaneously into SCID/bg mice. Twenty-four hours after the cell injection mice started to get treatment with subcutaneous injections of anti-human CCL20 antibody or isotype control, 20 μg of antibody per injection, three times a week, during four weeks. Significant decrease in tumor growth was observed in anti-CCL20-treated mice (FIG. 3E, 3F). In the control group nine out of ten mice developed subcutaneous tumors which rapidly progressed over time. In contrast, in anti-CCL20-treated group only five out of ten mice developed tumors, while four of produced tumors were very small in size (0.2 cm×0.2 cm or 0.1 cm×0.1 cm) and did not progress over time. Moreover, histological evaluation of H&E-stained tissue sections of xenograft tumors demonstrated that neutralizing antibodies to CCL20 inhibited intensive aberrant blood vessel formation and promoted extensive necrotic tissue damage in treated tumors.

To confirm the role of CCL20 in development and progression of CXCR4-overexpressing prostate tumors, the effect of neutralizing antibodies to human CCL20 on the growth of CXCR4-overexpressing PC3 cells was evaluated. PC3-CXCR4.5 cells were injected subcutaneously into SCID/bg mice. Injected animals were treated with the anti-human CCL20 antibody or isotype control according to the same protocol, as mice injected with CCL20-overexpressing cells. Animals treated with anti-CCL20 antibodies demonstrated a delay in tumor appearance—in the control group on day 28, 100 percents of the animals developed visible tumors, while in the anti-CCL20-treated group only 60 percents appeared with tumors on the same day. The treatment with anti-CCL20 antibodies inhibited the growth of CXCR4-expressing prostate tumors (FIG. 3G).

To further test the role of CCL20 in development and progression of tumors, the effect of neutralizing antibodies to human CCL20 on the growth of colon cancer HT-29 cells was evaluated. HT-29 cells were injected subcutaneously into nude mice. Injected animals were daily treated with the anti-human CCL20 antibody or isotype control. Treatment with anti-CCL20 antibodies significantly inhibited the growth and invasion of HT-29 tumors (FIG. 3H). Antibodies to CCL20 inhibited the invasion of tumor cells into surrounding tissues as the skin and muscle.

Thus, neutralizing antibodies to CCL20 suppressed in vivo CXCR4-dependent and independent prostate and colon tumor growth. These results also support the important role of CCL20 in CXCR4 dependent tumor development and progression.

### Example 4

### CXCR4 and CCL20 are Co Expressed in Human Prostate Cancer

In order to further test the relevance of in vivo role of CCL20/CCR6 axis in prostate cancer development, the expression of CCL20 and CCR6 in human prostate cancer tissues was first evaluated with the use of commercially available array of 52 paraffin-embedded prostate sections from patients with advanced prostate cancer. All specimens were graded using pathologic stage and the Gleason score system.

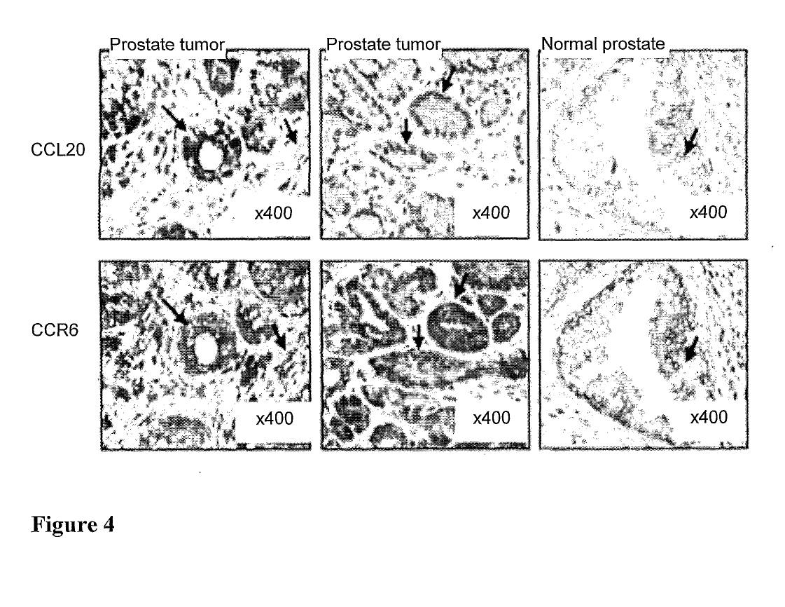

The immunohistochemical staining revealed that the majority of tumor samples (50 out of 52, 96%) expressed the CCR6 receptor at heterogeneous levels, whereas the ligand for CCR6, CCL20, was expressed in 34 out of 52 tumor samples (65.4%). The CCL20 and CCR6 staining were located mostly in epithelial and fibromuscular stromal cells (FIG. 4A). The majority of tumor samples that expressed CCL20 co-expressed CCR6. In contrast, normal human prostate tissue samples expressed very low levels of CCL20 and CCR6 (FIG. 4A).

Table 1 depicts the results of a commercially available array including 52 samples (CA2) from patients with prostate cancer was stained for CCL20 (left) and CCR6 (right). Expression was scored at two levels: low or negative expression, and high expression. Statistical analysis of the immunohistochemical staining was performed using two-tailed Mann-Whitney test.

TABLE 1
CCL20, CCR6 and CXCR4 expression in prostate cancer samples

| CCL20 | Gleason | | CCR6 | Gleason | |
|---|---|---|---|---|---|
| expression | score | Stage | expression | score | Stage |
| Low mean | 7.72 ± 1.75 | 3.28 ± 0.97 | Low mean | 8.6 ± 1.58 | 3.5 ± 0.85 |
| N | 36 | 36 | N | 10 | 10 |
| High mean | 8.75 ± 1.48 | 3.75 ± 0.68 | High mean | 7.9 ± 1.75 | 3.4 ± 0.94 |
| N | 16 | 16 | N | 42 | 42 |
| Total mean | 8.04 ± 1.73 | 3.42 ± 0.91 | Total mean | 8.04 ± 1.73 | 3.42 ± 0.91 |
| N | 52 | 52 | N | 52 | 52 |
| Mann- | 174.500 | 214.000 | Mann- | 154.000 | 203.500 |
| Whitney U | 0.02 | 0.07 | Whitney U | 0.17 | 0.85 |
| Asymp Sig | | | Asymp Sig | | |
| (2-tailed) | | | (2-tailed) | | |

Out of 34 CCL20-positive sections, 16 samples were highly positive for CCL20. Average Gleason score in samples with high CCL20 expression was 8.75±1.48 (n=16) versus 7.72±1.75 (n=36) in samples with low or negative CCL20 expression (p=0.02) (Table 1, left). Average stage in samples with high CCL20 expression was 3.28±0.97 (n=16) versus 3.75±0.68 (n=36) in samples with low or negative CCL20 expression (p<=0.07) (Table 1, left). No prevalence in high levels of CCR6 in progressive stage 1V sections was detected (FIG. 4B, right). These results demonstrate that high CCL20 expression correlates with high Gleason score (e.g., tumor grade) and higher staging of the disease in this array. In order to further test the possible interaction between CXCR4 receptor and CCL20 chemokine in prostate cancer, the coexpression of CCL20 and CXCR4 was evaluated in human prostate cancer tissues. Similar expression level of CXCR4 and CCL20 was observed on the majority of prostate tumor samples (38 out of 48 samples, 79.5%). Four additional samples demonstrated close expression pattern, and only six samples out of 48 (12.5%) demonstrated different expression levels of CXCR4 and CCL20. These results suggest that in human prostate cancer CCR6, CCL20, and CXCR4 are commonly overexpressed and that there is a correlation between CXCR4 and CCL20 expression.

### REFERENCES

Barash et al., Radiology 243, 727-35 (June, 2007).
Bell et al., J Exp Med 190, 1417-26 (Nov. 15, 1999).
Bonnotte, M. Crittenden, N. Larmonier, M. Gough, and R. G. Vile, J Immunol 173, 4929-35 (Oct. 15, 2004).
Charbonnier et al., J Exp Med 190, 1755-68 (Dec. 20, 1999).
Darash-Yahana et al., FASEB J 18, 1240-2 (August, 2004).
Dieu et al., J Exp Med 188, 373-86 (Jul. 20, 1998).
Fushimi et al., J Clin Invest 105, 1383-93 (May, 2000).
Hieshima et al., J Biol Chem 272, 5846-53 (Feb. 28, 1997).
Iwasaki and Kelsall, J Exp Med 191, 1381-94 (Apr. 17, 2000).
Kleeff et al., hit J Cancer 81, 650-7 (May 17, 1999).
Muller et al., Nature 410, 50-6 (Mar. 1, 2001).
Rubie et al., Scand J Immunol 63, 468-77 (June, 2006).
Tanaka et al., Eur J Immunol 29, 633-42 (February, 1999).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for treating a CCL20 dependent cancer in a subject in need thereof, comprising administering to the subject a therapeutically effective amount of a pharmaceutical composition comprising a CCL20-neutralizing antibody.

2. The method according to claim 1 for inhibiting tumor growth or tumor progression in the subject.

3. The method according to claim 1 for inhibiting tumor vascularization in the subject.

4. The method according to claim 1 for inducing or enhancing tumor regression in the subject.

5. The method according to claim 1, wherein at least a portion of the cells of the cancer express CCR6.

6. The method according to claim 1, wherein at least a portion of the cells of the cancer express CXCR4.

7. The method according to claim 1, wherein at least a portion of the cells of the cancer are characterized by surface expression of CCR6 and CXCR4.

8. The method according to claim 1, wherein the cancer is selected from glioma, leukemia, uterine cancer, lymphoma, neuroblastoma, pancreatic cancer, prostate cancer, clear cell renal carcinoma, colon cancer, colorectal cancer, lung cancer, breast cancer and melanoma.

9. The method of claim 8, wherein the cancer is prostate cancer.

10. The method of claim 8, wherein the cancer is colon cancer.

11. The method according to claim 1, wherein the antibody is MAB360.

12. The method according to claim 1, wherein the antibody has the same specificity as MAB360, or wherein said antibody has at least an antigen-binding fragment of MAB360.

13. The method according to claim 1, wherein the antibody is administered to the subject by injection or infusion.

14. A method of inhibiting, preventing or reducing metastasis of a CCL20 dependent tumor in a subject in need thereof, comprising administering to a subject in need thereof a therapeutically effective amount of a CCL20-neutralizing antibody.

15. The method of claim 14, wherein the subject has a tumor expressing CCR6 and/or CXCR4 on at least a portion of the cells of the tumor.

16. The method according to claim 14, wherein the antibody is MAB360.

17. The method according to claim 14, wherein the antibody has the same specificity as MAB360, or wherein said antibody has at least an antigen-binding fragment of MAB360.

18. The method according to claim 14, wherein the antibody is administered to the subject by injection or infusion.

19.-24. (canceled)

25. A kit comprising a CCL20 neutralizing antibody, optionally formulated with at least one pharmaceutically acceptable excipient, and instructions for administering the antibody to a subject afflicted with cancer

26. The kit of claim 25, wherein the cancer is a CCL20 dependent cancer.

27. The kit of claim 26, wherein the cancer is selected from glioma, leukemia, uterine cancer, lymphoma, neuroblastoma, pancreatic cancer, prostate cancer, clear cell renal carcinoma, colorectal cancer, lung cancer, breast cancer and melanoma.

28. A kit according to claim 25, wherein the antibody is MAB360, an antibody having the same specificity as MAB360, or an antibody having at least an antigen-binding fragment of MAB360.
